(19) United States
(12) Patent Application Publication
(10) Pub. No.: US 2024/0207401 A1
(43) Pub. Date: Jun. 27, 2024

(54) CORONAVIRUS ANTIBODY

(71) Applicant: IMPERIAL COLLEGE INNOVATIONS LIMITED, London (GB)

(72) Inventors: Xiao-Ning Xu, London (GB); Hantao Lou, London (GB); Jia Guo, London (GB)

(73) Assignee: IMPERIAL COLLEGE INNOVATIONS LIMITED, London (GB)

(21) Appl. No.: 17/911,257

(22) PCT Filed: Mar. 19, 2021
(86) PCT No.: PCT/GB2021/050685

§ 371 Date: Feb. 16, 2023

(30) Foreign Application Priority Data
Mar. 19, 2020 (GB) 2003980.6
Nov. 26, 2020 (GB) 2018582.3

(51) Int. Cl. A61K 39/42; A61K 39/00; A61P 31/14; C07K 16/10; G01N 33/569; G01N 33/68

(52) U.S. Cl. CPC A61K 39/42; A61P 31/14; C07K 16/1003; G01N 33/56983; G01N 33/6854; A61K 2039/505; A61K 2039/70; G01N 2333/165

(57) ABSTRACT

An antibody or antigen-binding fragment thereof is described which binds to the spike protein S2 domain of severe acute respiratory syndrome coronavirus (SARS-CoV), Middle East respiratory syndrome-related coronavirus (MERS-CoV) and/or severe acute respiratory syndrome coronavirus 2 (SARS CoV-2). The antibodies can be used in the diagnosis and treatment of coronavirus infection. The invention extends to compositions comprising the antibodies, including pharmaceutical compositions, diagnostic compositions, and kits. The invention also extends to methods of making and using the antibodies, for example in the diagnosis and therapy of coronavirus infections.

### CROSS-REFERENCE TO RELATED APPLICATIONS

This application is a National Phase of International Application No. PCT/GB2021/050685, filed Mar. 19, 2021, which designated the U.S. and that International Application was published under PCT Article 21(2) in English. This application also includes a claim of priority under 35 U.S.C. § 119(a) and § 365(b) to British patent application Numbers 2003980.6, filed Mar. 19, 2020, and 2018582.3, filed Nov. 26, 2020, the entirety of all which are hereby incorporated by reference in their entirety.

### REFERENCE TO SEQUENCE LISTING

This application contains a Sequence Listing submitted as .txt file named “Sequence listing_ST25”, having a size in bytes of 77,853 bytes, and created on Feb. 14, 2023. The information contained in this electronic file is hereby incorporated by reference in its entirety.

The invention relates to antibodies, and in particular, to antibodies used in the diagnosis and treatment of coronavirus infection. The invention extends to compositions comprising the antibodies, including pharmaceutical compositions, diagnostic compositions, and kits. The invention also extends to methods of making and using the antibodies, for example in the diagnosis and therapy of coronavirus infections.

The severe acute respiratory syndrome coronavirus (SARS-CoV-2), causing 2019-nCoV or COVID-19 disease, is a virus belonging in the coronavirus (CoV) group of disease-causing pathogens that includes severe acute respiratory syndrome coronavirus (SARS) and Middle East respiratory syndrome-related coronavirus (MERS). Coronaviruses are usually restricted to their wild hosts (e.g. bats). However, both SARS and MERS, and more recently SARS-CoV-2, have all been transferred to humans, and this caused the SARS and MERS outbreaks of 2003, 2012, and 2019 respectively.

The coronavirus family has been identified recently in a number of emerging pathogen priority lists, i.e. UKVN, WHO blueprint and CEPI, highlighting the urgent need to improve our understanding of immune responses to coronaviruses, both to control current problems and also to be prepared for emerging threats. The large number of genetically distinct coronaviruses and increasing interface between human populations and animal reservoirs of CoV suggests that there is a significant risk of new CoV zoonotic infections. Indeed, the outbreak of SARS-CoV-2 in China has proved this to be the case. Coronaviruses tend to target the respiratory systems and so, due to these periodic outbreaks, a treatment effectively targeting coronaviruses is urgently needed. In addition, improved robust detection methods are also required.

No specific treatment is available for human CoV infections. However, monoclonal antibodies (mAbs) are increasingly being introduced as immunotherapeutic treatments against cancer and viral infections. Whilst they can have immunomodulatory abilities, in viral infections (e.g. HIV, HBV, HBC, and Ebola), they are mainly being used as neutralising agents due to their ability to bind, with high specificity, to viral surface antigens required for viral entry into the host cell. Antiviral mAbs can directly blunt viral propagation and, in some cases, engage the host's immune system, leading to long-lasting protective vaccine-like effects. However, because of the highly diversified nature of CoVs, most human mAbs isolated from patients are strain-specific, and no broadly neutralising antibody has been identified to date.

Therefore, the development of CoV-mAb-based immunotherapies for the highly pathogenic CoVs will address an immediate unmet medical need and could prove a rapid diagnosis and treatment, not only for current SARS-CoV-2, but also for future emerging pandemic CoVs.

Accordingly, in a first aspect of the invention, there is provided an antibody or antigen-binding fragment thereof that binds to the spike protein S2 domain of severe acute respiratory syndrome coronavirus (SARS-CoV), Middle East respiratory syndrome-related coronavirus (MERS-CoV) and/or severe acute respiratory syndrome coronavirus 2 (SARS-CoV-2).

The data described in the Examples show that the antibodies of the invention, by binding to the S2 domain of spike protein, display surprisingly robust cross-reactivity with multiple coronavirus strains. In addition, surprisingly, these antibodies display neutralising activity against each virus strain. The inventors believe that they are the first to develop antibodies that advantageously display cross-reactivity to SARS-CoV-2, MERS-CoV and SARS-CoV S, thereby providing a pan or ‘universal’ coronavirus antibody that can be used for detecting and treating all known coronavirus pathogens and possibly provide a means for detecting and treating future strains of coronavirus infections.

The skilled person would understand that SARS-CoV-2 may also be referred to as SARS CoV2, Coronavirus disease 2019 (COVID-19) or novel coronavirus (2019-nCoV).

Preferably, the antibody or antigen-binding fragment thereof is cross-reactive with SARS-CoV, MERS-CoV and SARS CoV2. Preferably, the antibody or antigen-binding fragment thereof neutralises SARS-CoV, MERS-CoV and SARS CoV2.

Preferably, the antibody or antigen-binding fragment thereof binds to the spike protein S2 domain of SARS-CoV and MERS-CoV. Preferably, the antibody or antigen-binding fragment thereof binds to the spike protein S2 domain of SARS-CoV and SARS CoV2. Preferably, the antibody or antigen-binding fragment thereof binds to the spike protein S2 domain of MERS-CoV and SARS CoV2.

Preferably, the antibody or antigen-binding fragment thereof binds to the spike protein S2 domain of a bat coronavirus variant that is similar to SARS-CoV. The bat coronavirus variant may be the WIV16 variant or the RaTG13 variant. Preferably, the antibody or antigen-binding fragment thereof binds to the spike protein S2 domain of a SARS-CoV-2 mink mutant. Preferably, the antibody or antigen-binding fragment thereof binds to the spike protein S2 domain of a SARS-CoV-2 B1.1.7 variant (“Kent variant”) or a SARS-CoV-2 B.1.351 variant (“South Africa variant”).

Most preferably, however, the antibody or antigen-binding fragment thereof binds to the spike protein S2 domain of SARS-CoV, MERS-CoV and SARS CoV2. Each of these S2 domains will now be described herein.

### SARS-CoV

In one embodiment, the SARS-CoV spike protein may be represented by Genbank ID No: AAR07630.1, which is provided herein as SEQ ID No: 1, as follows:

[SEQ ID No: 1]
MFIFLLFLTLTSGSDLDRCTTFDDVQAPNYTQHTSSMRGVYYPDEIFRSD
TLYLTQDLFLPFYSNVTGFHTINHTFGNPVIPFKDGIYFAATEKSNVVRG
WVFGSTMNNKSQSVIIINNSTNVVIRACNFELCDNPFFAVSKPMGTQTHT
MIFDNAFNCTFEYISDAFSLDVSEKSGNFKHLREFVFKNKDGFLYVYKGY
QPIDVVRDLPSGFNTLKPIFKLPLGINITNFRAILTAFSPAQDIWGTSAA
AYFVGYLKPTTFMLKYDENGTITDAVDCSQNPLAELKCSVKSFEIDKGIY
QTSNFRVVPSGDVVRFPNITNLCPFGEVFNATKFPSVYAWERKKISNCVA
DYSVLYNSTFFSTFKCYGVSATKLNDLCFSNVYADSFVVKGDDVRQIAPG
QTGVIADYNYKLPDDFMGCVLAWNTRNIDATSTGNYNYKYRYLRHGKLRP
FERDISNVPFSPDGKPCTPPALNCYWPLNDYGFYTTTGIGYQPYRVVVLS
FELLNAPATVCGPKLSTDLIKNQCVNFNFNGLTGTGVLTPSSKRFQPFQQ
FGRDVSDFTDSVRDPKTSEILDISPCSFGGVSVITPGTNASSEVAVLYQD
VNCTDVSTAIHADQLTPAWRIYSTGNNVFQTQAGCLIGAEHVDTSYECDI
PIGAGICASYHTVSLLRSTSQKSIVAYTMSLGADSSIAYSNNTIAIPTNF
SISITTEVMPVSMAKTSVDCNMYICGDSTECANLLLQYGSFCTQLNRALS
GIAAEQDRNTREVFAQVKQMYKTPTLKYFGGFNFSQILPDPLKPTKRSFI
EDLLFNKVTLADAGFMKQYGECLGDINARDLICAQKFNGLTVLPPLLTDD
MIAAYTAALVSGTATAGWTFGAGAALQIPFAMQMAYRFNGIGVTQNVLYE
NQKQIANQFNKAISQIQESLTTTSTALGKLQDVVNQNAQALNTLVKQLSS
NFGAISSVLNDILSRLDKVEAEVQIDRLITGRLQSLQTYVTQQLIRAAEI
RASANLAATKMSECVLGQSKRVDFCGKGYHLMSFPQAAPHGVVFLHVTYV
PSQERNFTTAPAICHEGKAYFPREGVFVFNGTSWFITQRNFFSPQIITTD
NTFVSGNCDVVIGIINNTVYDPLQPELDSFKEELDKYFKNHTSPDVDLGD
ISGINASVVNIQKEIDRLNEVAKNLNESLIDLQELGKYEQYIKWPWYVWL
GFIAGLIAIVMVTILLCCMTSCCSCLKGACSCGSCCKFDEDDSEPVLKGV
KLHYT

The antibody or antigen-binding fragment thereof may bind to a region between amino acid positions 700 and 900, 750 and 900, 800 and 900, 700 and 850, 750 and 850, 800 and 850, 700 and 800, or 750 and 800 of the SARS-CoV spike protein substantially as set out in SEQ ID No:1.

The antibody or antigen-binding fragment thereof may bind to a region between amino acid positions 770 and 790, 780 and 800, 790 and 810, or 800 and 820 of the SARS-CoV spike protein substantially as set out in SEQ ID No:1.

The antibody or antigen-binding fragment thereof may bind to a region between amino acid positions 774 and 790, 774 and 800, 774 and 810, 774 and 819, 780 and 819, 790 and 819, 800 and 819, or 810 and 819 of the SARS-CoV spike protein substantially as set out in SEQ ID No:1.

Preferably, however, the antibody or antigen-binding fragment thereof may bind to a region between amino acid positions 774 and 819 of the SARS-CoV spike protein substantially as set out in SEQ ID No:1.

In one embodiment, the SARS-CoV spike protein S2 domain may be represented by Genbank ID No: AAT74874.1, which is provided herein as SEQ ID No: 2, as follows:

[SEQ ID No: 2]
ASYHTVSLLRSTSQKSIVAYTMSLGADSSIAYSNNTIAIPTNFSISITTE
VMPVSMAKTSVDCNMYICGDSTECANLLLQYGSFCTQLNRALSGIAAEQD
RNTREVFAQVKQMYKTPTLKYFGGFNFSQILPDPLKPTKRSFIEDLLFNK
VTLADAGFMKQYGECLGDINARDLICAQKFNGLTVLPPLLTDDMIAAYTA
ALVSGTATAGWTFGAGAALQIPFAMQMAYRFNGIGVTQNVLYENQKQIAN
QFNKAISQIQESLTTTSTALGKLQDVVNQNAQALNTLVKQLSSNFGAISS
VLNDILSRLDKVEAEVQIDRLITGRLQSLQTYVTQQLIRAAEIRASANLA
ATKMSECVLGQSKRVDFCGKGYHLMSFPQAAPHGVVFLHVTYVPSQERNF
TTAPAICHEGKAYFPREGVFVFNGTSWFITQRNFFSPQIITTDNTFVSGN
CDVVIGIINNTVYDPLQPELDSFKEELDKYFKNHTSPDVDLGDISGINAS
VVNIQKEIDRLNEVAKNLNESLIDLQELGKYEQYIKWPWYVWLGFIAGLI
AIVMVTILLCCMTSCCSCLKGACSCGSCCKFDEDDSEPVLKGVKL

Preferably, the antibody or antigen-binding fragment thereof binds to a sequence comprising or consisting of a sequence as substantially set out in SEQ ID No: 2, or a fragment or variant thereof.

Preferably, the antibody or antigen-binding fragment thereof binds to one or more amino acid in SEQ ID No: 2, or a fragment or variant thereof.

Preferably, the antibody or antigen-binding fragment thereof binds to any 5, 10, 15, 20, 25, 30, 35, 40 or 45 amino acids present in SEQ ID No: 2, or a variant or fragment thereof.

Preferably, the antibody or antigen-binding fragment thereof may bind to one or more amino acid between amino acid positions 774 and 819 of the SARS-CoV spike protein, which is provided herein as SEQ ID No: 3, as follows:

[SEQ ID No: 3]
PTLKYFGGFNFSQILPDPLKPTKRSFIEDLLFNKVTLADAGFMKQY

Thus, preferably the antibody or antigen-binding fragment thereof binds to a sequence comprising or consisting of a sequence as substantially set out in SEQ ID No: 3, or a variant or fragment thereof.

Preferably, the antibody or antigen-binding fragment thereof binds to one or more amino acid in SEQ ID No: 3, or a fragment or variant thereof.

Preferably, the antibody or antigen-binding fragment thereof binds to any 5, 10, 15, 20, 25, 30, 35, 40 or 45 amino acid sequence present in SEQ ID No: 3, or a variant or fragment thereof.

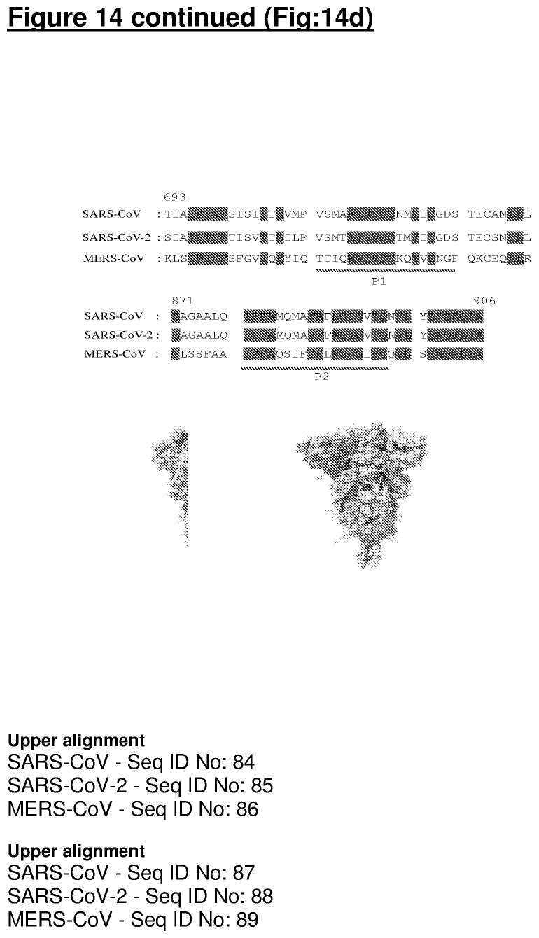

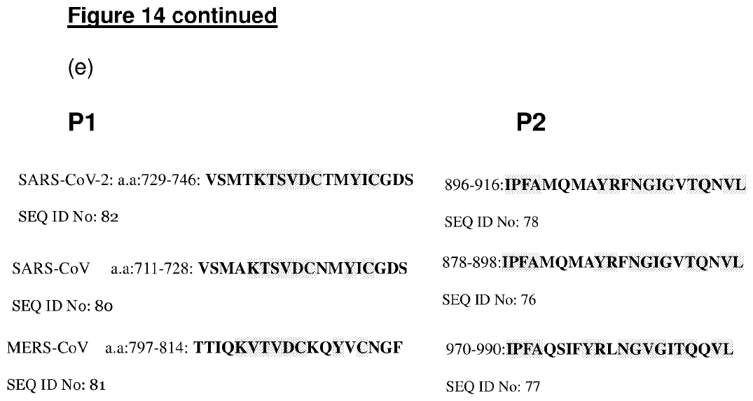

As described in the Examples, and as illustrated in FIG. 14c-e, the inventors found that the antibody or antigen-binding fragment thereof according to the invention binds to one or more epitope (herein denoted P1 and P2) in the spike protein S2 domain in each of SARS-CoV, MERS-CoV and/or SARS-CoV-2. Accordingly, below are described the P1 and P2 epitopes for SARS-CoV.

In a preferred embodiment, therefore, the antibody or antigen-binding fragment thereof may bind to a region between amino acid positions 690 and 950 of the SARS-CoV spike protein substantially as set out in SEQ ID No: 1. The antibody or antigen-binding fragment thereof may bind to a region between amino acid positions 710 and 910 of the SARS-CoV spike protein substantially as set out in SEQ ID No: 1. The antibody or antigen-binding fragment thereof may bind to a region between amino acid positions 690 and 750, 700 and 740, or 711 and 730 of the SARS-CoV spike protein substantially as set out in SEQ ID No:1. This epitope is denoted P1, as shown in FIGS. 14d and e. The antibody or antigen-binding fragment thereof may bind to a region between amino acid positions 705 and 735, 710 and 730, 711 and 740, 711 and 730, 700 and 728, 710 and 728, or 711 and 728 of the SARS-CoV spike protein substantially as set out in SEQ ID No:1. Preferably, the antibody or antigen-binding fragment thereof may bind to a region between amino acid positions 711 and 728 of the SARS-CoV spike protein substantially as set out in SEQ ID No:1.

The antibody or antigen-binding fragment thereof may bind to a region between amino acid positions 840 and 930, 850 and 920, 860 and 910, or 870 and 900 of the SARS-CoV spike protein substantially as set out in SEQ ID No:1. This epitope is denoted P2, as shown in FIGS. 14d and e. The antibody or antigen-binding fragment thereof may bind to a region between amino acid positions 865 and 898, 870 and 898, 878 and 910, 878 and 905, or 878 and 900 of the SARS-CoV spike protein substantially as set out in SEQ ID No:1. Preferably, the antibody or antigen-binding fragment thereof may bind to a region between amino acid positions 878 and 898 of the SARS-CoV spike protein substantially as set out in SEQ ID No:1.

Thus, in one embodiment, the antibody or antigen-binding fragment thereof may bind to a region between amino acid positions 711 and 728, and/or between 878 and 898 of the SARS-CoV spike protein substantially as set out in SEQ ID No:1.

Preferably, the antibody or antigen-binding fragment thereof may bind to one or more amino acid between amino acid positions 711 and 728 of the SARS-CoV spike protein (i.e. the P1 epitope), which is provided herein as SEQ ID No: 80, as follows:

[SEQ ID No: 80]
VSMAKTSVDCNMYICGDS

Thus, preferably the antibody or antigen-binding fragment thereof binds to a sequence comprising or consisting of a sequence as substantially set out in SEQ ID No: 80, or a variant or fragment thereof.

Preferably, the antibody or antigen-binding fragment thereof binds to one or more amino acid in SEQ ID No: 80, or a fragment or variant thereof.

Preferably, the antibody or antigen-binding fragment thereof binds to any 5, 10, 15 or 20 amino acid sequence present in SEQ ID No: 80, or a variant or fragment thereof.

Preferably, the antibody or antigen-binding fragment thereof may bind to one or more amino acid between amino acid positions 878 and 898 of the SARS-CoV spike protein (i.e. the P2 epitope), which is provided herein as SEQ ID No: 76, as follows:

[SEQ ID No: 76]
IPFAMQMAYRFNGIGVTQNVL

Thus, preferably the antibody or antigen-binding fragment thereof binds to a sequence comprising or consisting of a sequence as substantially set out in SEQ ID No: 76, or a variant or fragment thereof.

Preferably, the antibody or antigen-binding fragment thereof binds to one or more amino acid in SEQ ID No: 76, or a fragment or variant thereof.

Preferably, the antibody or antigen-binding fragment thereof binds to any 5, 10, 15 or 20 amino acid sequence present in SEQ ID No: 76, or a variant or fragment thereof.

### MERS-CoV

In one embodiment, the MERS-CoV spike protein may be represented by Genbank ID No: AHX71946.1, which is provided herein as SEQ ID No: 4, as follows:

[SEQ ID No: 4]
MIHSVFLLMFLLTPTESYVDVGPDSVKSACIEVDIQQTFFDKTWPRPIDV
SKADGIIYPQGRTYSNITITYQGLFPYQGDHGDMYVYSAGHATGTTPQKL
FVANYSQDVKQFANGFVVRIGAAANSTGTVIISPSTSATIRKIYPAFMLG
SSVGNFSDGKMGRFFNHTLVLLPDGCGTLLRAFYCILEPRSGNHCPAGNS
HTSFATYHTPATDCSDGNYNRNASLNSFKEYFNLRNCTFMYTYNITEDEI
LEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCS
YESFDVESGVYSVSSFEAKPSGSVVEQAEGVECDFSPLLSGTPPQVYNFK
RLVFTNCNYNLTKLLSLFSVNDFTCSQISPAAIASNCYSSLILDYFSYPL
SMKSDLSVSSAGPISQFNYKQSFSNPTCLILATVPHNLTTITKPLKYSYI
NKCSRLLSDDRTEVPQLVNANQYSPCVSIVPSTVWEDGDYYRKQLSPLEG
GGWLVASGSTVAMTEQLQMGFGITVQYGTDTNSVCPKLEFANDTKIASQL
GNCVEYSLYGVSGRGVFQNCTAVGVRQQRFVYDAYQNLVGYYSDDGNYYC
LRACVSVPVSVIYDKETKTHATLFGSVACEHISSTMSQYSRSTRSMLKRR
DSTYGPLQTPVGCVLGLVNSSLFVEDCKLPLGQSLCALPDTPSTLTPRSV
RSVPGEMRLASIAFNHPIQVDQLNSSYFKLSIPTNFSFGVTQEYIQTTIQ
KVTVDCKQYVCNGFQKCEQLLREYGQFCSKINQALHGANLRQDDSVRNLF
ASVKSSQSSPIIPGFGGDFNLTLLEPVSISTGSRSARSAIEDLLFDKVTI
ADPGYMQGYDDCMQQGPASARDLICAQYVAGYKVLPPLMDVNMEAAYTSS
LLGSIAGVGWTAGLSSFAAIPFAQSIFYRLNGVGITQQVLSENQKLIANK
FNQALGAMQTGFTTTNEAFRKVQDAVNNNAQALSKLASELSNTFGAISAS
IGDIIQRLDVLEQDAQIDRLINGRLTTLNAFVAQQLVRSESAALSAQLAK
DKVNECVKAQSKRSGFCGQGTHIVSFVVNAPNGLYFMHVGYYPSNHIEVV
SAYGLCDAANPTNCIAPVNGYFIKTNNTRIVDEWSYTGSSFYAPEPITSL
NTKYVAPQVTYQNISTNLPPPLLGNSTGIDFQDELDEFFKNVSTSIPNFG
SLTQINTTLLDLTYEMLSLQQVVKALNESYIDLKELGNYTYYNKWPWYIW
LGFIAGLVALALCVFFILCCTGCGTNCMGKLKCNRCCDRYEEYDLEPHKV
HVH

The antibody or antigen-binding fragment thereof may bind to a region between amino acid positions 800 and 1000, 800 and 950, 800 and 900, 850 and 1000, 850 and 950, 850 and 900, or 900 and 950 of the MERS-CoV spike protein substantially as set out in SEQ ID No:4.

The antibody or antigen-binding fragment thereof may bind to a region between amino acid positions 850 and 870, 860 and 880, 870 and 890 or 880 and 900, or 890 and 910 of the MERS-CoV spike protein substantially as set out in SEQ ID No:4.

The antibody or antigen-binding fragment thereof may bind to a region between amino acid positions 860 and 880, 860 and 890, 860 and 900, 860 and 910, 870 and 910, 880 and 910, 890 and 910, or 900 and 910 of the MERS-CoV spike protein substantially as set out in SEQ ID No:4.

Preferably, the antibody or antigen-binding fragment thereof may bind to a region between amino acid positions 860 and 910 of the MERS-CoV spike protein substantially as set out in SEQ ID No:4.

In one embodiment, the MERS-CoV spike protein S2 domain may be represented by Genbank ID No: ALA49836.1, which is provided here in as SEQ ID No: 5, as follows:

[SEQ ID No: 5]
VDQLNSSYFKLSIPTNFSFGVTQEYIQTTIQKVTVDCKQYVCNGFQKCEQ
LLREYGQFCSKINQALHGANLRQDDSVRNLFASVKSSQSSPIIPGFGGDF
NLTLLEPVSISTGSRSARSAIEDLLFDKVTIADPGYMQGYDDCMQQGPAS
ARDLICAQYVAGYKVLPPLMDVNMEAAYTSSLLGSIAGVGWTAGLSSFAA
IPFAQSIFYRINGVGITQQVLSENQKLIANKFNQALGAMQTGFTTTNEAF
RKVQDAVNNNAQALSKLASELSNTFGAISASIGDIIQRLDVLEQDAQIDR
LINGRLTTLNAFVAQQLVRSESAALSAQLAKDKVNECVKAQSKRSGFCGQ
GTHIVSFVVNAPNGLYFMHVGYYPSNHIEVVSAYGLCDAANPTNCIAPVN
GYFIKTNNTRIVDEWSYTGSSFYAPEPITSLNTKYVAPQVTYQNISTNLP
PPLLGNSTGIDFQDELDEFFKNVSTSIPNFGSLTQINTTLLDLTYEMLSL
QQVVKALNESYIDLKELGNYTYYNKWPWYIWLGFIAGLVALALCVFFILC
CTGCGTNCMGKLKCNRCCDRYEEYDLEPHKVH

Preferably, the antibody or antigen-binding fragment thereof binds to a sequence comprising or consisting of a sequence as substantially set out in SEQ ID No: 5, or a fragment or variant thereof.

Preferably, the antibody or antigen-binding fragment thereof binds to one or more amino acid in SEQ ID No: 5, or a fragment or variant thereof.

Preferably, the antibody or antigen-binding fragment thereof binds to any 5, 10, 15, 20, 25, 30, 35, 40 or 45 amino acid sequence present in SEQ ID No: 5, or a variant or fragment thereof.

The antibody or antigen-binding fragment thereof may bind to one or more amino acid between amino acid positions 860 and 910 of the MERS-CoV spike protein, which is provided herein as SEQ ID No: 6, as follows:

[SEQ ID No: 6]
PIIPGFGGDFNLTLLEPVSISTGSRSARSAIEDLLFDKVTIADPGYMQGY

Thus, preferably, the antibody or antigen-binding fragment thereof binds to a sequence comprising or consisting of a sequence as substantially set out in SEQ ID No: 6, or a variant or fragment thereof.

Preferably, the antibody or antigen-binding fragment thereof binds to one or more amino acid in SEQ ID No: 6, or a fragment or variant thereof.

Preferably, the antibody or antigen-binding fragment thereof binds to any 5, 10, 15, 20, 25, 30, 35, 40 or 45 amino acid sequence present in SEQ ID No: 6, or a variant or fragment thereof.

With reference to FIG. 14c-e, the inventors found that the antibody or antigen-binding fragment thereof according to the invention binds to one or more epitope (herein denoted P1 and P2) in the spike protein S2 domain in MERS-CoV, as follows.

Preferably, the antibody or antigen-binding fragment thereof may bind to a region between amino acid positions 760 and 1150, 770 and 1100, 780 and 1000, or 790 and 1000 of the MERS-CoV spike protein substantially as set out in SEQ ID No:4. The antibody or antigen-binding fragment thereof may bind to a region between amino acid positions 760 and 850, 770 and 840, 780 and 830, 790 and 820, or 795 and 815 of the MERS-CoV spike protein substantially as set out in SEQ ID No:4. This epitope is denoted P1, as shown in FIGS. 14d and e. Preferably, the antibody or antigen-binding fragment thereof may bind to a region between amino acid positions 797 and 814 of the MERS-CoV spike protein substantially as set out in SEQ ID No: 4.

The antibody or antigen-binding fragment thereof may bind to a region between amino acid positions 940 and 1020, 950 and 1010, 960 and 1000, or 970 and 995 of the MERS-CoV spike protein substantially as set out in SEQ ID No:4. This epitope is denoted P2, as shown in FIGS. 14d and e. Preferably, the antibody or antigen-binding fragment thereof may bind to a region between amino acid positions 970 and 990 of the MERS-CoV spike protein substantially as set out in SEQ ID No:4.

Thus, in one embodiment, the antibody or antigen-binding fragment thereof may bind to a region between amino acid positions 797 and 814 and/or between 970 and 990 of the MERS-CoV spike protein substantially as set out in SEQ ID No:4.

The antibody or antigen-binding fragment thereof may bind to one or more amino acid between amino acid positions 797 and 814 of the MERS-CoV spike protein (i.e. the P1 epitope), which is provided herein as SEQ ID No: 81, as follows:

[SEQ ID No: 81]
TTIQKVTVDCKQYVCNGF

Thus, preferably, the antibody or antigen-binding fragment thereof binds to a sequence comprising or consisting of a sequence as substantially set out in SEQ ID No: 81, or a variant or fragment thereof.

Preferably, the antibody or antigen-binding fragment thereof binds to one or more amino acid in SEQ ID No: 81, or a fragment or variant thereof.

Preferably, the antibody or antigen-binding fragment thereof binds to any 5, 10, 15 or 20 amino acid sequence present in SEQ ID No: 81, or a variant or fragment thereof.

The antibody or antigen-binding fragment thereof may bind to one or more amino acid between amino acid positions 970-990 of the MERS-CoV spike protein (i.e. the P2 epitope), which is provided herein as SEQ ID No: 77, as follows:

[SEQ ID No: 77]
IPFAQSIFYRLNGVGITQQVL

Thus, preferably, the antibody or antigen-binding fragment thereof binds to a sequence comprising or consisting of a sequence as substantially set out in SEQ ID No: 77, or a variant or fragment thereof.

Preferably, the antibody or antigen-binding fragment thereof binds to one or more amino acid in SEQ ID No: 77, or a fragment or variant thereof.

Preferably, the antibody or antigen-binding fragment thereof binds to any 5, 10, 15 or 20 amino acid sequence present in SEQ ID No: 77, or a variant or fragment thereof.

### SARS-CoV-2 (COVID-19)

In one embodiment, the SARS-CoV2 spike protein may be represented by Genbank ID No: YP009724390.1, which is provided herein as SEQ ID No: 7, as follows:

[SEQ ID No: 7]
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGENCYFPLQSYGFQPT
NGVGYQPYRVVVLSFELLHAPATVCGPKKSTNLVKNKCVNFNENGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLENKVTLADAGFIKQYGDCLGDIAARDLI
CAQKENGLTVLPPLLTDEMIAQYTSALLAGTITSGWTFGAGAALQIPFAM
QMAYRENGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

The antibody or antigen-binding fragment thereof may bind to a region between amino acid positions 700 and 900, 750 and 900, 800 and 900, 700 and 850, 750 and 850, 800 and 850, 700 and 800, or 750 and 800 of the SARS-CoV2 spike protein substantially as set out in SEQ ID No:7.

The antibody or antigen-binding fragment thereof may bind to a region between amino acid positions 790 and 810, 800 and 820, 810 and 830, or 820 and 840 of the SARS-CoV2 spike protein substantially as set out in SEQ ID No:7.

The antibody or antigen-binding fragment thereof may bind to a region between amino acid positions 792 and 800, 792 and 810, 792 and 820, 792 and 830, 800 and 837, 810 and 837, 820 and 837, 830 and 837 substantially as set out in SEQ ID No:7.

Preferably, the antibody or antigen-binding fragment thereof may bind to a region between amino acid positions 792 and 837 of the SARS-CoV2 spike protein substantially as set out in SEQ ID No:7.

In one embodiment, the SARS-CoV2 spike protein S2 domain may be represented by Genbank ID No: YP009724390.1, which is provided herein as SEQ ID No: 8, as follows:

[SEQ ID No: 8]
VASQSIIAYTMSLGAENSVAYSNNSIAIPTNFTISVTTEILPVSMTKTSV
DCTMYICGDSTECSNLLLQYGSFCTQLNRALTGIAVEQDKNTQEVFAQVK
QIYKTPPIKDFGGFNFSQILPDPSKPSKRSFIEDLLFNKVTLADAGFIKQ
YGDCLGDIAARDLICAQKFNGLTVLPPLLTDEMIAQYTSALLAGTITSGW
TFGAGAALQIPFAMQMAYRENGIGVTQNVLYENQKLIANQFNSAIGKIQD
SLSSTASALGKLQDVVNQNAQALNTLVKQLSSNFGAISSVLNDILSRLDK
VEAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNNT
VYDPLQPELDSFKEELDKYFKNHTSPDVDLGDISGINASVVNIQKEIDRL
NEVAKNLNESLIDLQELGKYEQYIKWPWYIWLGFIAGLIAIVMVTIMLCC
MTSCCSCLKGCCSCGSCCKFDEDDSEPVLKGVKLHYT*

Preferably, the antibody or antigen-binding fragment thereof binds to a sequence comprising or consisting of a sequence as substantially set out in SEQ ID No: 8, or a fragment or variant thereof.

Preferably, the antibody or antigen-binding fragment thereof binds to one or more amino acid in SEQ ID No: 8, or a fragment or variant thereof.

Preferably, the antibody or antigen-binding fragment thereof binds to any 5, 10, 15, 20, 25, 30, 35, 40 or 45 amino acid sequence present in SEQ ID No: 8, or a variant or fragment thereof.

Thus, the antibody or antigen-binding fragment thereof may bind to one or more amino acid between amino acid positions 792 and 837 of the SARS-CoV2 spike protein, which is provided herein as SEQ ID No: 9, as follows:

[SEQ ID No: 9]
PPIKDFGGFNFSQILPDPSKPSKRSFIEDLLFNKVTLADAGFIKQY

Thus, preferably, the antibody or antigen-binding fragment thereof binds to a sequence comprising or consisting of a sequence as substantially set out in SEQ ID No: 9, or a variant or fragment thereof.

Preferably, the antibody or antigen-binding fragment thereof binds to one or more amino acid in SEQ ID No: 9, or a fragment or variant thereof.

Preferably, the antibody or antigen-binding fragment thereof binds to any 5, 10, 15, 20, 25, 30, 35, 40 or 45 amino acid sequence present in SEQ ID No: 9, or a variant or fragment thereof.

With reference to FIG. 14c-e, the inventors found that the antibody or antigen-binding fragment thereof according to the invention binds to one or more epitope (herein denoted P1 and P2) in the spike protein S2 domain in SARS-CoV2, as follows.

Preferably, the antibody or antigen-binding fragment thereof may bind to a region between amino acid positions 700 and 950, 800 and 950, 850 and 950, 700 and 900, 700 and 850, or 700 and 800 of the SARS-CoV2 spike protein substantially as set out in SEQ ID No: 7.

The antibody or antigen-binding fragment thereof may bind to a region between amino acid positions 690 and 780, 700 and 770, 710 and 760, or 720 and 750 of the SARS-CoV2 spike protein substantially as set out in SEQ ID No: 7. This epitope is denoted P1, as shown in FIGS. 14d and e. The antibody or antigen-binding fragment thereof may bind to a region between amino acid positions 729 and 770, 729 and 760, 729 and 750, 700 and 746, 710 and 746, 720 and 746, or 725 and 746 of the SARS-CoV2 spike protein substantially as set out in SEQ ID No: 7. Preferably, the antibody or antigen-binding fragment thereof may bind to a region between amino acid positions 729 and 746 of the SARS-CoV2 spike protein substantially as set out in SEQ ID No: 7.

The antibody or antigen-binding fragment thereof may bind to a region between amino acid positions 870 and 930, 880 and 925, 890 and 920, 896 and 930, 896 and 925, 896 and 920, 880 and 916, 885 and 916, or 890 and 916 of the SARS-CoV2 spike protein substantially as set out in SEQ ID No:7. This epitope is denoted P2, as shown in FIGS. 14d and e. Preferably, the antibody or antigen-binding fragment thereof may bind to a region between amino acid positions 896 and 916 of the SARS-CoV2 spike protein substantially as set out in SEQ ID No:7.

Thus, in one embodiment, the antibody or antigen-binding fragment thereof may bind to a region between amino acid positions 729 and 746, and/or between 896 and 916 of the SARS-CoV2 spike protein substantially as set out in SEQ ID No:7.

Thus, the antibody or antigen-binding fragment thereof may bind to one or more amino acid between amino acid positions 729 and 746 of the SARS-CoV2 spike protein (i.e. the P1 epitope), which is provided herein as SEQ ID No: 82, as follows:

[SEQ ID No: 82]
VSMTKTSVDCTMYICGDS

Thus, preferably, the antibody or antigen-binding fragment thereof binds to a sequence comprising or consisting of a sequence as substantially set out in SEQ ID No: 82, or a variant or fragment thereof.

Preferably, the antibody or antigen-binding fragment thereof binds to one or more amino acid in SEQ ID No: 82, or a fragment or variant thereof.

Preferably, the antibody or antigen-binding fragment thereof binds to any 5, 10, 15 and 20 amino acid sequence present in SEQ ID No: 82, or a variant or fragment thereof.

The antibody or antigen-binding fragment thereof may bind to one or more amino acid between amino acid positions 896 and 916 of the SARS-CoV2 spike protein (i.e. the P2 epitope), which is provided herein as SEQ ID No: 78, as follows:

[SEQ ID No: 78]
IPFAMQMAYRFNGIGVTQNVL

Thus, preferably, the antibody or antigen-binding fragment thereof binds to a sequence comprising or consisting of a sequence as substantially set out in SEQ ID No: 78, or a variant or fragment thereof.

Preferably, the antibody or antigen-binding fragment thereof binds to one or more amino acid in SEQ ID No: 78, or a fragment or variant thereof.

Preferably, the antibody or antigen-binding fragment thereof binds to any 5, 10, 15 and 20 amino acid sequence present in SEQ ID No: 9, or a variant or fragment thereof.

### Consensus Sequences

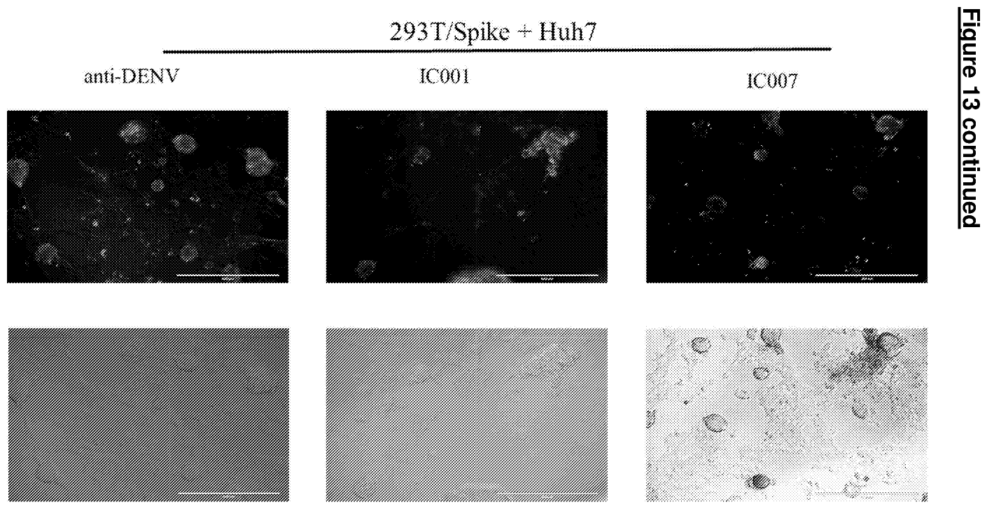

Based on the inventor's identification of a shared epitope region in the S2 domain of spike protein of SARS-CoV, MER-CoV and SARS-CoV2, as shown in FIG. 12, the inventors have been able to generate a consensus epitope sequence for cross-reactive antibody binding, which is provided herein as SEQ ID No: 10, as follows:

[SEQ ID No: 10]
PX₁X₂X₃X₄FGGX₃FNX₅X₆X₇X₈X₉PX₁₀X₁₁X₁₂X₁₃X₁₄X₁₅X₁₆X₁₇X₁₈X₁₉R
SX₂₀IEDLLFX₂₁KVTX₂₂ADX₂₃GX₂₄X₂₅X₂₆X₂₇Y

Preferably, therefore, the antibody or antigen-binding fragment thereof binds to a sequence comprising or consisting of a sequence as substantially set out in SEQ ID No: 10, or a fragment or variant thereof, in which X₁ to X₂₇ can be any amino acid or no amino acid. Preferably, X₁ to X₄, X₆ to X₁₁ and X₁₄ to X₂₇ can be any amino acid and X₅, X₁₁ to X₁₃ can be no amino acid.

X₁ to X₂₇ may be an amino acid that is present in the corresponding amino position in the MERS-CoV sequence of SEQ ID No: 6, SARS-CoV sequence of SEQ ID No: 3 or SARS-CoV-2 sequence of SEQ ID No: 9.

In a preferred embodiment, however, and based on the inventor's identification of a shared epitope region (i.e. the P1 epitope) in the S2 domain of spike protein of SARS-CoV, MER-CoV and SARS-CoV2, as shown in FIG. 14d, the inventors have been able to generate a consensus epitope sequence for cross-reactive antibody binding, which is provided herein as SEQ ID No: 83, as follows:

[SEQ ID No: 83]
X₁X₂X₃X₄KTSVDCX₅X₆YX₇CX₈X₉X₁₀

Preferably, therefore, the antibody or antigen-binding fragment thereof binds to a sequence comprising or consisting of a sequence as substantially set out in SEQ ID No: 83, or a fragment or variant thereof, in which X₁ to X₁₀ can be any amino acid.

X₁ to X₁₀ may be an amino acid that is present in the corresponding amino position in the MERS-CoV sequence of SEQ ID No: 6, SARS-CoV sequence of SEQ ID No: 3 or SARS-CoV-2 sequence of SEQ ID No: 9.

The inventors have identified a further shared epitope region (i.e. the P2 epitope), as shown in FIG. 14d, and the inventors have been able to generate a further consensus epitope sequence for cross-reactive antibody binding, which is provided herein as SEQ ID No: 79, as follows:

[SEQ ID No: 79]
IPFAX₁X₂X₃X₄YRX₅NGIGX₆TQX₇VL

Preferably, therefore, the antibody or antigen-binding fragment thereof binds to a sequence comprising or consisting of a sequence as substantially set out in SEQ ID No: 79, or a fragment or variant thereof, in which X₁ to X₇ can be any amino acid.

X₁ to X₇ may be an amino acid that is present in the corresponding amino position in the MERS-CoV sequence of SEQ ID No: 77, SARS-CoV sequence of SEQ ID No: 76 or SARS-CoV-2 sequence of SEQ ID No: 78.

Preferably, the antibody or antigen binding fragment thereof of the invention is capable of neutralising and/or inducing antibody-dependent cellular cytotoxicity (ADCC) of SARS-CoV, MERS-CoV and/or SARS CoV2.

The skilled person would appreciate that “neutralising” means reducing or neutralising the biological effect of the virus. Preferably, the antibody is capable of blocking spike protein from binding to angiotensin-converting enzyme 2 (ACE2) present on the cell membrane in a host cell.

Preferably, the antibody or antigen binding fragment thereof of the invention is capable of eliciting an immune response against SARS-CoV, MERS-CoV and/or SARS CoV2.

Preferably, the antibody or antigen binding fragment thereof stimulates production of neutralizing antibodies and/or both CD4+ and CD8+ T cell responses in a host.

The invention extends to both whole antibodies (i.e. immunoglobulins) with immunospecificity for the S2 domain of SARS-CoV, MERS-CoV and/or SARS CoV2, as well as to antigen-binding fragments or regions of the corresponding full-length antibody.

The antibody or antigen-binding fragment thereof may be monovalent, divalent or polyvalent. Monovalent antibodies are dimers (HL) comprising a heavy (H) chain associated by a disulphide bridge with a light chain (L). Divalent antibodies are tetramer (H2L2) comprising two dimers associated by at least one disulphide bridge. Polyvalent antibodies may also be produced, for example by linking multiple dimers.

The basic structure of an antibody molecule consists of two identical light chains and two identical heavy chains which associate non-covalently and can be linked by disulphide bonds. Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen, i.e. the S2 domain of SARS-CoV, MERS-CoV and/or SARS CoV2, or variant or fragment thereof (e.g. an epitope). On either side of the CDRs of the heavy and light chains is a framework region, a relatively conserved sequence of amino acids that anchors and orients the CDRs. Antibody fragments may include a bi-specific antibody (BsAb) or a chimeric antigen receptor (CAR).

The heavy chain constant region typically comprises three domains, C_{H1}, C_{H2}, and C_{H3}. Each light chain typically comprises a light chain variable region (V_{1.}) and a light chain constant region. The light chain constant region typically comprises one domain, abbreviated C_{1.}.

Each heavy chain and light chain generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. See Kabat et al., Sequences of Proteins of Immunological Interest 5th ed. (1991) Public Health Service, National Institutes of Health, Bethesda, MD, incorporated by reference in its entirety.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The light chain from any vertebrate species can be assigned to one of two types, called kappa and lambda, based on the sequence of the constant domain.

The constant region consists of one of five heavy chain sequences (μ, γ, ζ, α, or ε) and one of two light chain sequences (κ or λ). The heavy chain constant region sequences determine the isotype of the antibody and the effector functions of the molecule.

Preferably, the antibody or antigen-binding fragment thereof is isolated or purified.

In one preferred embodiment, the antibody or antigen-binding fragment thereof comprises a polyclonal antibody, or an antigen-binding fragment thereof. The antibody or antigen-binding fragment thereof may be generated in a rabbit, mouse or rat.

Preferably, the antibody or antigen-binding fragment thereof is obtained by immunising a host animal with the S2 domain of SARS-CoV, MERS-CoV and/or SARS CoV2, or a variant or fragment thereof, and then collecting the antibody or antigen-binding fragment thereof. The host animal is most preferably a rabbit.

In another preferred embodiment, the antibody or antigen-binding fragment thereof comprises a monoclonal antibody or an antigen-binding fragment thereof. Preferably, the antibody of the invention is a human antibody. As used herein, the term “human antibody” can mean an antibody, such as a monoclonal antibody, which comprises substantially the same heavy and light chain CDR amino acid sequences as found in a particular human antibody exhibiting immunospecificity for the S2 domain of SARS-CoV, MERS-CoV and/or SARS CoV2, or a variant or fragment thereof. An amino acid sequence, which is substantially the same as a heavy or light chain CDR, exhibits a considerable amount of sequence identity when compared to a reference sequence. Such identity is definitively known or recognizable as representing the amino acid sequence of the particular human antibody. Substantially the same heavy and light chain CDR amino acid sequence can have, for example, minor modifications or conservative substitutions of amino acids. Such a human antibody maintains its function of selectively binding to the S2 domain of SARS-CoV, MERS-CoV and/or SARS CoV2 or a variant or fragment thereof.

The term “human monoclonal antibody” can include a monoclonal antibody with substantially or entirely human CDR amino acid sequences produced, for example by recombinant methods such as production by a phage library, by lymphocytes or by hybridoma cells.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “humanised antibody” can mean an antibody from a non-human species (e.g. mouse or rabbit) whose protein sequences have been modified to increase their similarity to antibodies produced naturally in humans.

The antibody may be a recombinant antibody. The term “recombinant human antibody” can include a human antibody produced using recombinant DNA technology.

The term “antigen-binding region” can mean a region of the antibody having specific binding affinity for its target antigen, for example, the S2 domain of SARS-CoV, MERS-CoV and/or SARS CoV2, or a variant or fragment thereof. Preferably, the fragment is an epitope. The antigen-binding region may be a hypervariable CDR or a functional portion thereof. The term “functional portion” of a CDR can mean a sequence within the CDR which shows specific affinity for the target antigen. The functional portion of a CDR may comprise a ligand which specifically binds to the S2 domain of SARS-CoV, MERS-CoV and/or SARS CoV2, or a fragment thereof.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The amino acid sequence boundaries of a CDR can be determined by using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme).

The term “functional fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab′; bivalent fragments such as F(ab′)2; single chain Fv (scFv); and Fc fragments.

The term “VL fragment” can mean a fragment of the light chain of a human monoclonal antibody which includes all or part of the light chain variable region, including the CDRs. A VL fragment can further include light chain constant region sequences.

The term “VH fragment” can mean a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-1. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about 110 to about 220 of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain.

The term “F(ab′)₂ fragment” can mean a bivalent antigen-binding fragment of a human monoclonal antibody. An F(ab′)₂ fragment includes, for example, all or part of the variable regions of two heavy chains- and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

One skilled in the art knows that the exact boundaries of a fragment of an antibody are not important, so long as the fragment maintains a functional activity. Using well-known recombinant methods, one skilled in the art can engineer a polynucleotide sequence to express a functional fragment with any endpoints desired for a particular application. A functional fragment of the antibody may comprise or consist of a fragment with substantially the same heavy and light chain variable regions as the human antibody.

Preferably, the antigen-binding fragment thereof, with respect to the first aspect of the invention, is immunospecific for an epitope within the S2 domain of SARS-CoV, MERS-CoV and/or SARS CoV2. The antigen-binding fragment thereof may comprise or consist of any of the fragments selected from a group consisting of VH, VL, Fd, Fv, Fab, Fab′, scFv, F (ab′)₂ and Fc fragment.

The antigen-binding fragment thereof may comprise or consist of any one of the antigen binding region sequences of the VL, any one of the antigen binding region sequences of the VH, or a combination of VL and VH antigen binding regions of a human antibody. The appropriate number and combination of VH and VL antigen binding region sequences may be determined by those skilled in the art depending on the desired affinity and specificity and the intended use of the antigen-binding fragment. Functional fragments or antigen-binding fragments of antibodies may be readily produced and isolated using methods well known to those skilled in the art. Such methods include, for example, proteolytic methods, recombinant methods and chemical synthesis. Proteolytic methods for the isolation of functional fragments comprise using human antibodies as a starting material. Enzymes suitable for proteolysis of human immunoglobulins may include, for example, papain, and pepsin. The appropriate enzyme may be readily chosen by one skilled in the art, depending on, for example, whether monovalent or bivalent fragments are required. For example, papain cleavage results in two monovalent Fab′ fragments that bind antigen and an Fc fragment. Pepsin cleavage, for example, results in a bivalent F (ab′) fragment. An F (ab′)₂ fragment of the invention may be further reduced using, for example, DTT or 2-mercaptoethanol to produce two monovalent Fab′ fragments.

Functional or antigen-binding fragments of antibodies produced by proteolysis may be purified by affinity and column chromatographic procedures. For example, undigested antibodies and Fc fragments may be removed by binding to protein A. Additionally, functional fragments may be purified by virtue of their charge and size, using, for example, ion exchange and gel filtration chromatography. Such methods are well known to those skilled in the art.

The antibody or antigen-binding fragment thereof may be produced by recombinant methodology. Preferably, one initially isolates a polynucleotide encoding desired regions of the antibody heavy and light chains. Such regions may include, for example, all or part of the variable region of the heavy and light chains. Preferably, such regions can particularly include the antigen binding regions of the heavy and light chains, preferably the antigen binding sites, most preferably the CDRs.

The polynucleotide encoding the antibody or antigen-binding fragment thereof according to the invention may be produced using methods known to those skilled in the art. The polynucleotide encoding the antibody or antigen-binding fragment thereof may be directly synthesized by methods of oligonucleotide synthesis known in the art. Alternatively, smaller fragments may be synthesized and joined to form a larger functional fragment using recombinant methods known in the art.

As used herein, the term “immunospecificity” can mean the binding region of the antibody or antigen-binding fragment thereof is capable of immunoreacting with the S2 domain of SARS-CoV, MERS-CoV and/or SARS CoV2, or a variant or fragment thereof, by specifically binding therewith. The antibody or antigen-binding fragment thereof can selectively interact with an antigen (S2 domain of SARS-CoV, MERS-CoV and/or SARS CoV2) with an affinity constant of approximately 10⁻⁵ to 10⁻¹³ M⁻¹, preferably 10⁻⁶ to 10⁻⁹ M⁻¹, even more preferably, 10⁻¹⁰ to 10⁻¹² M⁻¹.

The term “immunoreact” can mean the binding region is capable of eliciting an immune response upon binding with the S2 domain of SARS-CoV, MERS-CoV and/or SARS CoV2, or an epitope thereof.

The term “epitope” can mean any region of an antigen with the ability to elicit, and combine with, a binding region of the antibody or antigen-binding fragment thereof. The epitope may be a linear or conformational epitope. Preferably, the epitope is linear. This can mean that the antibody interacts with a plurality of continuous amino acids of the antigen, and so the epitope can consist of these defined amino acids. Preferably, the epitope is conformational, i.e. non-linear or discontinuous. This can mean that the antibody interacts with multiple, distinct segments from the primary amino acid sequence of the antigen. The epitope may comprise the epitope denoted P1 and/or the epitope denoted P2, as defined herein.

Thus, the antibody the antibody or antigen-binding fragment thereof may comprise a heavy chain. The heavy chain may be selected from the group consisting of IgA; IgD; IgE; IgG and IgM. Preferably, the heavy chain is an IgG. Preferably, the heavy chain is an IgA.

The heavy chain may be an IgG1. The heavy chain may be an IgG2. The heavy chain may be an IgG3. The heavy chain may be an IgG4. The heavy chain may be an IgA1. The heavy chain may be an IgA2.

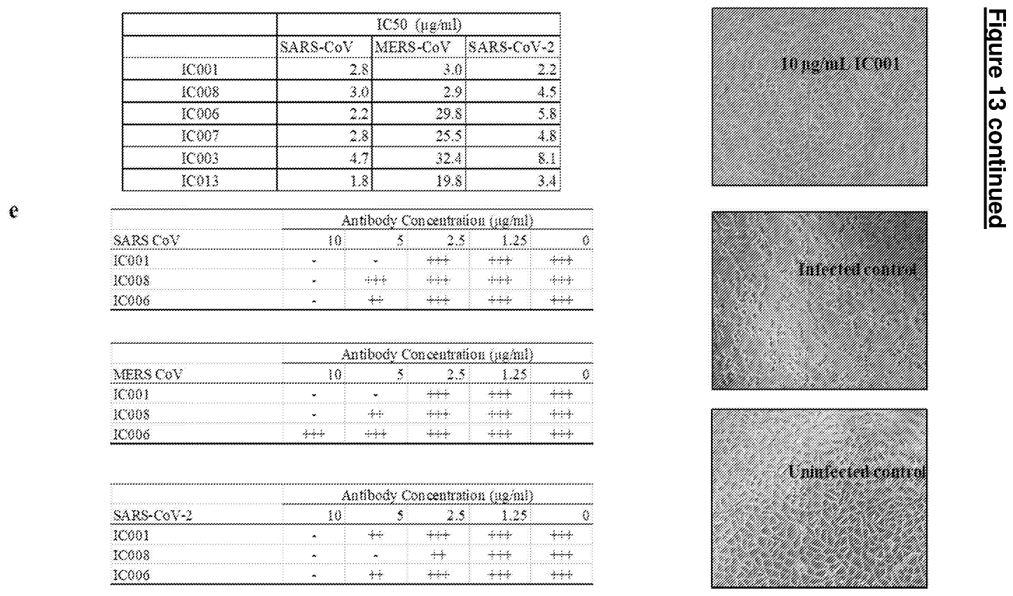

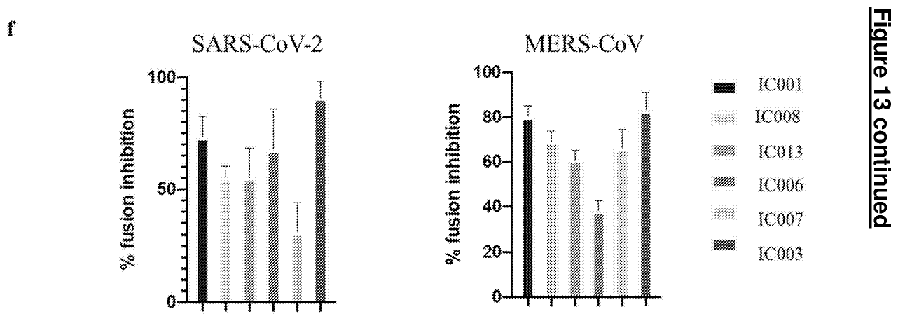

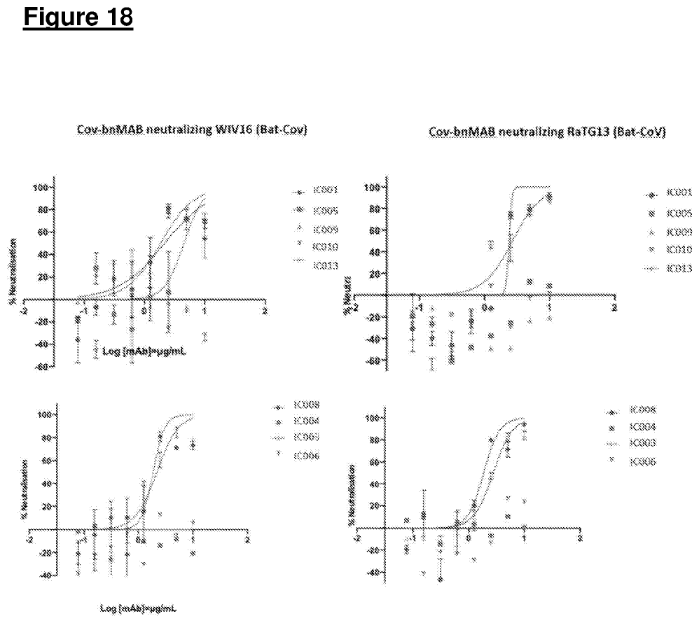

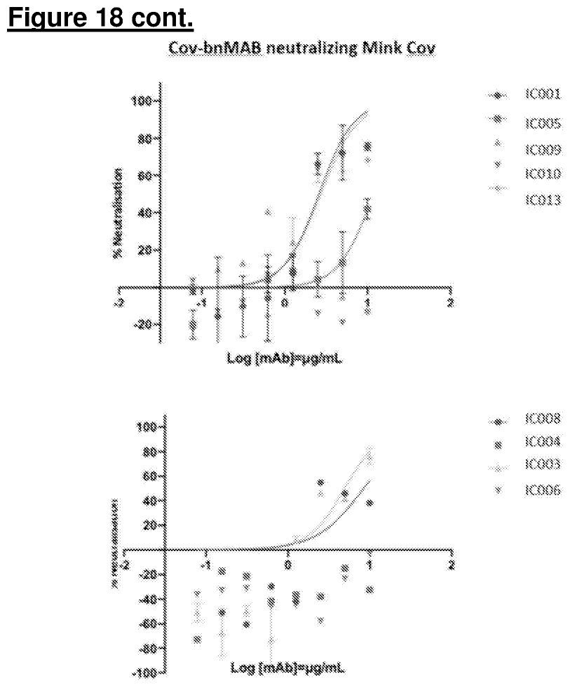

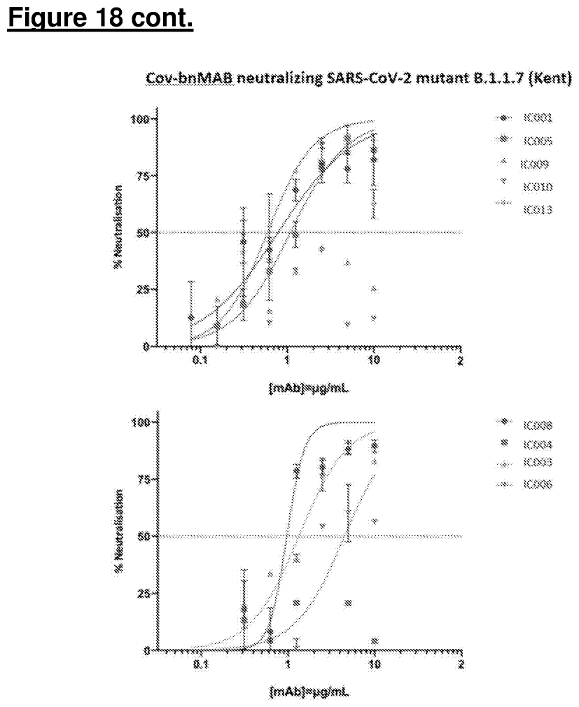

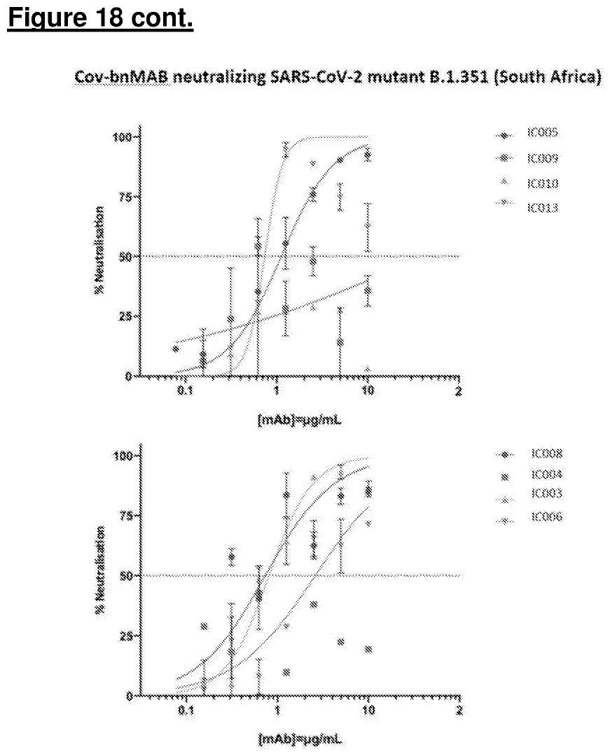

As described in the Examples, and as shown in FIG. 18, the inventors have isolated 16 antibodies which are highly immunospecific for SARS-CoV, 15 antibodies which are immunospecific for MERS-CoV, and nine antibodies which are immunospecific for SARS CoV2. The inventors have also advantageously identified four monoclonal antibodies that display surprisingly high cross-reactivity with SARS-CoV, MERS-CoV and SARS CoV2. These antibodies are identified herein as IC001, IC008, IC006 and IC004, and their CDR and framework sequences for the heavy and light chains are summarised in FIG. 11, and as discussed in detail below.

### IC001

In one embodiment, the antibody or antigen-binding fragment thereof is referred to herein as IC001 (see line 1 of the tables shown in FIGS. 11A and B). The antibody or antigen-binding fragment thereof may comprise a CDR-H1 domain of SEQ ID No: 11, which is provided herein, as follows:

[SEQ ID No: 11]
GYTFTSYA

Thus, preferably, the antibody or antigen-binding fragment thereof comprises a CDR-H1 domain comprising or consisting of a sequence as substantially set out in SEQ ID No: 11, or a variant or fragment thereof.

The antibody or antigen-binding fragment thereof may comprise a CDR-H2 domain of SEQ ID No: 12, which is provided herein, as follows:

[SEQ ID No: 12]
INAGNGNT

Thus, preferably the antibody or antigen-binding fragment thereof comprises a CDR-H2 domain comprising or consisting of a sequence as substantially set out in SEQ ID No: 12, or a variant or fragment thereof.

The antibody or antigen-binding fragment thereof may comprise a CDR-H3 domain of SEQ ID No: 13, which is provided herein, as follows:

[SEQ ID No: 13]
ARDRHMVVPAAVFDY

Thus, preferably the antibody or antigen-binding fragment thereof comprises a CDR-H3 domain comprising or consisting of a sequence as substantially set out in SEQ ID No: 13, or a variant or fragment thereof.

Preferably, the antibody or antigen-binding fragment thereof comprises a CDR-H1 domain comprising or consisting of SEQ ID No: 11, a CDR-H2 domain comprising or consisting of SEQ ID No: 12 and/or a CDR-H3 domain comprising or consisting of SEQ ID No: 13. Preferably, however, the antibody or antigen-binding fragment thereof comprises a CDR-H1 domain comprising or consisting of SEQ ID No: 11, a CDR-H2 domain comprising or consisting of SEQ ID No: 12 and a CDR-H3 domain comprising or consisting of SEQ ID No: 13.

The antibody or antigen-binding fragment thereof may comprise a FR-H1 domain of SEQ ID No: 14, which is provided herein, as follows:

[SEQ ID No: 14]
QVQLVQSGAEVKKPGASVKVSCKAS

Thus, preferably the antibody or antigen-binding fragment thereof comprises a FR-H1 domain comprising or consisting of a sequence as substantially set out in SEQ ID No: 14, or a variant or fragment thereof.

The antibody or antigen-binding fragment thereof may comprise a FR-H2 domain of SEQ ID No: 15, which is provided herein, as follows:

[SEQ ID No: 15]
MHWVRQAPGQRLEWMGW

Thus, preferably the antibody or antigen-binding fragment thereof comprises a CDR-FR-H2 domain comprising or consisting of a sequence as substantially set out in SEQ ID No: 15, or a variant or fragment thereof.

The antibody or antigen-binding fragment thereof may comprise a FR-H3 domain of SEQ ID No: 16, which is provided herein, as follows:

[SEQ ID No: 16]
KYSQKFQGRVTITRDTSASTAYMELSSLRSEDTAVYYC

Thus, preferably the antibody or antigen-binding fragment thereof comprises a CDR-FR-H3 domain comprising or consisting of a sequence as substantially set out in SEQ ID No: 16, or a variant or fragment thereof.

The antibody or antigen-binding fragment thereof may comprise a FR-H4 domain of SEQ ID No: 17, which is provided herein, as follows:

[SEQ ID No: 17]
WGQGTLVTVSS

Thus, preferably the antibody or antigen-binding fragment thereof comprises a FR-H4 domain comprising or consisting of a sequence as substantially set out in SEQ ID No: 17, or a variant or fragment thereof.

Preferably, the antibody or antigen-binding fragment thereof comprises a FR-H1 domain comprising or consisting of SEQ ID No: 14, a FR-H2 domain comprising or consisting of SEQ ID No: 15, a FR-H3 domain comprising or consisting of SEQ ID No: 16 and/or a FR-H4 domain comprising or consisting of SEQ ID No: 17. Preferably, however, the antibody or antigen-binding fragment thereof comprises a FR-H1 domain comprising or consisting of SEQ ID No: 14, a FR-H2 domain comprising or consisting of SEQ ID No: 15, a FR-H3 domain comprising or consisting of SEQ ID No: 16 and a FR-H4 domain comprising or consisting of SEQ ID No: 17.

The antibody or antigen-binding fragment thereof may comprise a heavy chain sequence as set out in SEQ ID No: 18, which is provided herein, as follows:

[SEQ ID No: 18]
QVQLVQSGAEVKKPGASVKVSCKASGYTFTSYAMHWVRQAPGQRLEW
MGWINAGNGNTKYSQKFQGRVTITRDTSASTAYMELSSLRSEDTAVY
YCARDRHMVVPAAVFDYWGQGTLVTVSS

Preferably, the antibody or antigen-binding fragment thereof comprises a heavy chain variable region comprising or consisting of a sequence as substantially set out in SEQ ID No: 18, or a variant or fragment thereof.

The antibody or antigen-binding fragment thereof may comprise a light chain CDR-L1 domain of SEQ ID No: 19, which is provided herein, as follows:

[SEQ ID No: 19]
QSISSW

Thus, preferably the antibody or antigen-binding fragment thereof comprises a CDR-L1 domain comprising or consisting of a sequence as substantially set out in SEQ ID No: 19, or a variant or fragment thereof.

The antibody or antigen-binding fragment thereof may comprise a CDR-L2 domain of SEQ ID No: 20, which is provided herein, as follows:

[SEQ ID No: 20]
KAS

Thus, preferably the antibody or antigen-binding fragment thereof comprises a CDR-L2 domain comprising or consisting of a sequence as substantially set out in SEQ ID No: 20, or a variant or fragment thereof.

The antibody or antigen-binding fragment thereof may comprise a CDR-L3 domain of SEQ ID No: 21, which is provided herein, as follows:

[SEQ ID No: 21]
QQYGT

Thus, preferably the antibody or antigen-binding fragment thereof comprises a CDR-L3 domain comprising or consisting of a sequence as substantially set out in SEQ ID No: 21, or a variant or fragment thereof.

Preferably, the antibody or antigen-binding fragment thereof comprises a CDR-L1 domain comprising or consisting of SEQ ID No: 19, a CDR-L2 domain comprising or consisting of SEQ ID No: 20, and/or a CDR-L3 domain comprising or consisting of SEQ ID No: 21. However, preferably the antibody or antigen-binding fragment thereof comprises a CDR-L1 domain comprising or consisting of SEQ ID No: 19, a CDR-L2 domain comprising or consisting of SEQ ID No: 20, and a CDR-L3 domain comprising or consisting of SEQ ID No: 21.

The antibody or antigen-binding fragment thereof may comprise a FR-L1 domain of SEQ ID No: 22, which is provided herein, as follows:

[SEQ ID No: 22]
AIQLTQSPSTLSASVGDRVTITCRAS

Thus, preferably the antibody or antigen-binding fragment thereof comprises a FR-L1 domain comprising or consisting of a sequence as substantially set out in SEQ ID No: 22, or a variant or fragment thereof.

The antibody or antigen-binding fragment thereof may comprise a FR-L2 domain of SEQ ID No: 23, which is provided herein, as follows:

[SEQ ID No: 23]
LAWYQQKPGKAPKLLIY

Thus, preferably the antibody or antigen-binding fragment thereof comprises a CDR-FR-L2 domain comprising or consisting of a sequence as substantially set out in SEQ ID No: 23, or a variant or fragment thereof.

The antibody or antigen-binding fragment thereof may comprise a FR-L3 domain of SEQ ID No: 24, which is provided herein, as follows:

[SEQ ID No: 24]
SLESGVPSRFSGSGSGTEFTLTISSLQPDDFATYYC

Thus, preferably the antibody or antigen-binding fragment thereof comprises a CDR-FR-L3 domain comprising or consisting of a sequence as substantially set out in SEQ ID No: 24, or a variant or fragment thereof.

The antibody or antigen-binding fragment thereof may comprise a FR-L4 domain of SEQ ID No: 25, which is provided herein, as follows:

[SEQ ID No: 25]
FGQGTKWISN

Thus, preferably the antibody or antigen-binding fragment thereof comprises a FR-L4 domain comprising or consisting of a sequence as substantially set out in SEQ ID No: 25, or a variant or fragment thereof.

Preferably, the antibody or antigen-binding fragment thereof comprises a FR-L1 domain comprising or consisting of SEQ ID No: 22, a FR-L2 domain comprising or consisting of SEQ ID No: 23, a FR-L3 domain comprising or consisting of SEQ ID No: 24 and/or a FR-L4 domain comprising or consisting of SEQ ID No: 25. Preferably, however, the antibody or antigen-binding fragment thereof comprises a FR-L1 domain comprising or consisting of SEQ ID No: 22, a FR-L2 domain comprising or consisting of SEQ ID No: 23, a FR-L3 domain comprising or consisting of SEQ ID No: 24 and a FR-L4 domain comprising or consisting of SEQ ID No: 25.

The antibody or antigen-binding fragment thereof may comprise a light chain sequence as set out in SEQ ID No: 26, which is provided herein, as follows:

[SEQ ID No: 26]
AIQLTQSPSTLSASVGDRVTITCRASQSISSWLAWYQQKPGKAPKLL
IYKASSLESGVPSRFSGSGSGTEFTLTISSLQPDDFATYYCQQYGTF
GQGTKWISN

Preferably, the antibody or antigen-binding fragment thereof comprises a light chain variable region comprising or consisting of a sequence as substantially set out in SEQ ID No: 26, or a variant or fragment thereof.

Preferably, the antibody or antigen-binding fragment thereof comprises a CDR-H1 domain comprising or consisting of SEQ ID No: 11, a CDR-H2 domain comprising or consisting of SEQ ID No: 12, a CDR-H3 domain comprising or consisting of SEQ ID No: 13, a FR-H1 domain comprising or consisting of SEQ ID No: 14, a FR-H2 domain comprising or consisting of SEQ ID No: 15, a FR-H3 domain comprising or consisting of SEQ ID No: 16 and a FR-H4 domain comprising or consisting of SEQ ID No: 17.

Preferably, the antibody or antigen-binding fragment thereof comprises a CDR-L1 domain comprising or consisting of SEQ ID No: 19, a CDR-L2 domain comprising or consisting of SEQ ID No: 20, a CDR-L3 domain comprising or consisting of SEQ ID No: 21, a FR-L1 domain comprising or consisting of SEQ ID No: 22, a FR-L2 domain comprising or consisting of SEQ ID No: 23, a FR-L3 domain comprising or consisting of SEQ ID No: 24 and a FR-L4 domain comprising or consisting of SEQ ID No: 25.

Preferably, the antibody or antigen-binding fragment thereof comprises a CDR-H1 domain comprising or consisting of SEQ ID No: 11, a CDR-H2 domain comprising or consisting of SEQ ID No: 12, a CDR-H3 domain comprising or consisting of SEQ ID No: 13, a CDR-L1 domain comprising or consisting of SEQ ID No: 19, a CDR-L2 domain comprising or consisting of SEQ ID No: 20, and/or a CDR-L3 domain comprising or consisting of SEQ ID No: 21.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 11, a CDR-H2 domain comprising or consisting of SEQ ID No: 12; a CDR-H3 domain comprising or consisting of SEQ ID No: 13, a CDR-L1 domain comprising or consisting of SEQ ID No: 19, a CDR-L2 domain comprising or consisting of SEQ ID No: 20, and a CDR-L3 domain comprising or consisting of SEQ ID No: 21.

Preferably, the antibody or antigen-binding fragment thereof comprises a heavy chain variable region comprising or consisting of SEQ ID No: 18 and a light chain variable region comprising or consisting of SEQ ID No: 26.

### IC008

In one embodiment, the antibody or antigen-binding fragment thereof is referred to herein as IC008 (see line 2 of the tables shown in FIGS. 11A and B). The antibody or antigen-binding fragment thereof may comprise a CDR-H1 domain of SEQ ID No: 27, which is provided herein, as follows:

[SEQ ID No: 27]
GGSISSSRHY

Thus, preferably the antibody or antigen-binding fragment thereof comprises a CDR-H1 domain comprising or consisting of a sequence as substantially set out in SEQ ID No: 27, or a variant or fragment thereof.

The antibody or antigen-binding fragment thereof may comprise a CDR-H2 domain of SEQ ID No: 28, which is provided herein, as follows:

[SEQ ID No: 28]
IDYSGGT

Thus, preferably the antibody or antigen-binding fragment thereof comprises a CDR-H2 domain comprising or consisting of a sequence as substantially set out in SEQ ID No: 28, or a variant or fragment thereof.

The antibody or antigen-binding fragment thereof may comprise a CDR-H3 domain of SEQ ID No: 29, which is provided herein, as follows:

[SEQ ID No: 29]
ARQVGHSGRGHNWFDP

Thus, preferably the antibody or antigen-binding fragment thereof comprises a CDR-H3 domain comprising or consisting of a sequence as substantially set out in SEQ ID No: 29, or a variant or fragment thereof.

The antibody or antigen-binding fragment thereof comprises a CDR-H1 domain comprising or consisting of SEQ ID No: 27, a CDR-H2 domain comprising or consisting of SEQ ID No: 28; and/or a CDR-H3 domain comprising or consisting of SEQ ID No: 29.

Preferably, the antibody or antigen-binding fragment thereof comprises a CDR-H1 domain comprising or consisting of SEQ ID No: 27, a CDR-H2 domain comprising or consisting of SEQ ID No: 28, and a CDR-H3 domain comprising or consisting of SEQ ID No: 29.

The antibody or antigen-binding fragment thereof may comprise a FR-H1 domain of SEQ ID No: 30, which is provided herein, as follows:

[SEQ ID No: 30]
LVQLQESGPRLVTPSETLSLTCTVS

Thus, preferably the antibody or antigen-binding fragment thereof comprises a FR-H1 domain comprising or consisting of a sequence as substantially set out in SEQ ID No: 30, or a variant or fragment thereof.

The antibody or antigen-binding fragment thereof may comprise a FR-H2 domain of SEQ ID No: 31, which is provided herein, as follows:

[SEQ ID No: 31]
WGWIRQPPGMGLEWIGS

Thus, preferably the antibody or antigen-binding fragment thereof comprises a FR-H2 domain comprising or consisting of a sequence as substantially set out in SEQ ID No: 31, or a variant or fragment thereof.

The antibody or antigen-binding fragment thereof may comprise a FR-H3 domain of SEQ ID No: 32, which is provided herein, as follows:

[SEQ ID No: 32]
YCNPSLKSRVTISEDTSKNQFSLKVNSVTAADTAVYYC

Thus, preferably the antibody or antigen-binding fragment thereof comprises a FR-H3 domain comprising or consisting of a sequence as substantially set out in SEQ ID No: 32, or a variant or fragment thereof.

The antibody or antigen-binding fragment thereof may comprise a FR-H4 domain of SEQ ID No: 33, which is provided herein, as follows:

[SEQ ID No: 33]
WGQGTLVTVSS

Thus, preferably the antibody or antigen-binding fragment thereof comprises a FR-H4 domain comprising or consisting of a sequence as substantially set out in SEQ ID No: 33, or a variant or fragment thereof.

Preferably, the antibody or antigen-binding fragment thereof comprises a FR-H1 domain comprising or consisting of SEQ ID No: 30, a FR-H2 domain comprising or consisting of SEQ ID No: 31, a FR-H3 domain comprising or consisting of SEQ ID No: 32 and/or a FR-H4 domain comprising or consisting of SEQ ID No: 33.

Preferably, the antibody or antigen-binding fragment thereof comprises a FR-H1 domain comprising or consisting of SEQ ID No: 30, a FR-H2 domain comprising or consisting of SEQ ID No: 31, a FR-H3 domain comprising or consisting of SEQ ID No: 32 and a FR-H4 domain comprising or consisting of SEQ ID No: 33.

The antibody or antigen-binding fragment thereof may comprise a heavy chain sequence as set out in SEQ ID No: 34, which is provided herein, as follows:

[SEQ ID No: 34]
LVQLQESGPRLVTPSETLSLTCTVSGGSISSSRHYWGWIRQPPGMGLEW
IGSIDYSGGTYCNPSLKSRVTISEDTSKNQFSLKVNSVTAADTAVYYCA
RQVGHSGRGHNWFDPWGQGTLVTVSS

Preferably, the antibody or antigen-binding fragment thereof comprises a heavy chain variable region comprising or consisting of a sequence as substantially set out in SEQ ID No: 34, or a variant or fragment thereof.

The antibody or antigen-binding fragment thereof may comprise a light chain CDR-L1 domain of SEQ ID No: 35, which is provided herein, as follows:

[SEQ ID No: 35]
QSIYNY

Thus, preferably the antibody or antigen-binding fragment thereof comprises a CDR-L1 domain comprising or consisting of a sequence as substantially set out in SEQ ID No: 35, or a variant or fragment thereof.

The antibody or antigen-binding fragment thereof may comprise a CDR-L2 domain of SEQ ID No: 36, which is provided herein, as follows:

[SEQ ID No: 36]
AAS

Thus, preferably the antibody or antigen-binding fragment thereof comprises a CDR-L2 domain comprising or consisting of a sequence as substantially set out in SEQ ID No: 36, or a variant or fragment thereof.

The antibody or antigen-binding fragment thereof may comprise a CDR-L3 domain of SEQ ID No: 37, which is provided herein, as follows:

[SEQ ID No: 37]
QQSYSSSVT

Thus, preferably the antibody or antigen-binding fragment thereof comprises a CDR-L3 domain comprising or consisting of a sequence as substantially set out in SEQ ID No: 37, or a variant or fragment thereof.

Preferably, the antibody or antigen-binding fragment thereof comprises a CDR-L1 domain comprising or consisting of SEQ ID No: 35, a CDR-L2 domain comprising or consisting of SEQ ID No: 36, and/or a CDR-L3 domain comprising or consisting of SEQ ID No: 37.

Preferably, the antibody or antigen-binding fragment thereof comprises a CDR-L1 domain comprising or consisting of SEQ ID No: 35, a CDR-L2 domain comprising or consisting of SEQ ID No: 36, and a CDR-L3 domain comprising or consisting of SEQ ID No: 37.

The antibody or antigen-binding fragment thereof may comprise a FR-L1 domain of SEQ ID No: 38, which is provided herein, as follows:

[SEQ ID No: 38]
DIQMTQSPSSLSASVGDRVTITCRAS

Thus, preferably the antibody or antigen-binding fragment thereof comprises a FR-L1 domain comprising or consisting of a sequence as substantially set out in SEQ ID No: 38, or a variant or fragment thereof.

The antibody or antigen-binding fragment thereof may comprise a FR-L2 domain of SEQ ID No: 39, which is provided herein, as follows:

[SEQ ID No: 39]
LNWYQQKPGKAPKFLIY

Thus, preferably the antibody or antigen-binding fragment thereof comprises a FR-L2 domain comprising or consisting of a sequence as substantially set out in SEQ ID No: 39, or a variant or fragment thereof.

The antibody or antigen-binding fragment thereof may comprise a FR-L3 domain of SEQ ID No: 40, which is provided herein, as follows:

[SEQ ID No: 40]
TLQTGVPSRFSGSGSGTDFTLTISSLQPEDFATYYC

Thus, preferably the antibody or antigen-binding fragment thereof comprises a FR-L3 domain comprising or consisting of a sequence as substantially set out in SEQ ID No: 40, or a variant or fragment thereof.

The antibody or antigen-binding fragment thereof may comprise a FR-L4 domain of SEQ ID No: 41, which is provided herein, as follows:

[SEQ ID No: 41]
FGQGTRLEIK

Thus, preferably the antibody or antigen-binding fragment thereof comprises a FR-L4 domain comprising or consisting of a sequence as substantially set out in SEQ ID No: 41, or a variant or fragment thereof.

Preferably, the antibody or antigen-binding fragment thereof comprises a FR-L1 domain comprising or consisting of SEQ ID No: 38, a FR-L2 domain comprising or consisting of SEQ ID No: 39, a FR-L3 domain comprising or consisting of SEQ ID No: 40 and/or a FR-L4 domain comprising or consisting of SEQ ID No: 41.

Preferably, the antibody or antigen-binding fragment thereof comprises a FR-L1 domain comprising or consisting of SEQ ID No: 38, a FR-L2 domain comprising or consisting of SEQ ID No: 39, a FR-L3 domain comprising or consisting of SEQ ID No: 40 and a FR-L4 domain comprising or consisting of SEQ ID No: 41.

The antibody or antigen-binding fragment thereof may comprise a light chain sequence as set out in SEQ ID No: 42, which is provided herein, as follows:

[SEQ ID No: 42]
DIQMTQSPSSLSASVGDRVTITCRASQSIYNYLNWYQQKPGKAPKFLIY
AASTLQTGVPSRFSGSGSGTDFTLTISSLQPEDFATYYCQQSYSSSVTF
GQGTRLEIK

Preferably, the antibody or antigen-binding fragment thereof comprises a light chain variable region comprising or consisting of a sequence as set out in SEQ ID No: 42, or a variant or fragment thereof.

Preferably, the antibody or antigen-binding fragment thereof comprises a CDR-H1 domain comprising or consisting of SEQ ID No: 27, a CDR-H2 domain comprising or consisting of SEQ ID No: 28, a CDR-H3 domain comprising or consisting of SEQ ID No: 29; a FR-H1 domain comprising or consisting of SEQ ID No: 30, a FR-H2 domain comprising or consisting of SEQ ID No: 31, a FR-H3 domain comprising or consisting of SEQ ID No: 32 and a FR-H4 domain comprising or consisting of SEQ ID No: 33.

Preferably, the antibody or antigen-binding fragment thereof comprises a CDR-L1 domain comprising or consisting of SEQ ID No: 35, a CDR-L2 domain comprising or consisting of SEQ ID No: 36, a CDR-L3 domain comprising or consisting of SEQ ID No: 37, a FR-L1 domain comprising or consisting of SEQ ID No: 38, a FR-L2 domain comprising or consisting of SEQ ID No: 39, a FR-L3 domain comprising or consisting of SEQ ID No: 40 and a FR-L4 domain comprising or consisting of SEQ ID No: 41.

Preferably, the antibody or antigen-binding fragment thereof comprises a CDR-H1 domain comprising or consisting of SEQ ID No: 27, a CDR-H2 domain comprising or consisting of SEQ ID No: 28, a CDR-H3 domain comprising or consisting of SEQ ID No: 29, a CDR-L1 domain comprising or consisting of SEQ ID No: 35, a CDR-L2 domain comprising or consisting of SEQ ID No: 36, and/or a CDR-L3 domain comprising or consisting of SEQ ID No: 37.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 27, a CDR-H2 domain comprising or consisting of SEQ ID No: 28, a CDR-H3 domain comprising or consisting of SEQ ID No: 29, a CDR-L1 domain comprising or consisting of SEQ ID No: 35, a CDR-L2 domain comprising or consisting of SEQ ID No: 36, and a CDR-L3 domain comprising or consisting of SEQ ID No: 37.

Preferably, the antibody or antigen-binding fragment thereof comprises a heavy chain variable region comprising or consisting of SEQ ID No: 34 and a light chain variable region comprising or consisting of SEQ ID No: 42.

### IC006

In one embodiment, the antibody or antigen-binding fragment thereof is referred to herein as IC006 (see line 3 of the tables shown in FIGS. 11A and B). The antibody or antigen-binding fragment thereof may comprise a CDR-H1 domain of SEQ ID No: 43, which is provided herein, as follows:

[SEQ ID No: 43]
GFTFSSYA

Thus, preferably the antibody or antigen-binding fragment thereof comprises a CDR-H1 domain comprising or consisting of a sequence as substantially set out in SEQ ID No: 43, or a variant or fragment thereof.

The antibody or antigen-binding fragment thereof may comprise a CDR-H2 domain of SEQ ID No: 44, which is provided herein, as follows:

[SEQ ID No: 44]
ISGSGGST

Thus, preferably the antibody or antigen-binding fragment thereof comprises a CDR-H2 domain comprising or consisting of a sequence as substantially set out in SEQ ID No: 44, or a variant or fragment thereof.

The antibody or antigen-binding fragment thereof may comprise a CDR-H3 domain of SEQ ID No: 45, which is provided herein, as follows:

[SEQ ID No: 45]
AKAGNSKLRFFDWLLTM

Thus, preferably the antibody or antigen-binding fragment thereof comprises a CDR-H3 domain comprising or consisting of a sequence as substantially set out in SEQ ID No: 45, or a variant or fragment thereof.

Preferably, the antibody or antigen-binding fragment thereof comprises a CDR-H1 domain comprising or consisting of SEQ ID No: 43, a CDR-H2 domain comprising or consisting of SEQ ID No: 44, and/or a CDR-H3 domain comprising or consisting of SEQ ID No: 45.

Preferably, the antibody or antigen-binding fragment thereof comprises a CDR-H1 domain comprising or consisting of SEQ ID No: 43, a CDR-H2 domain comprising or consisting of SEQ ID No: 44, and a CDR-H3 domain comprising or consisting of SEQ ID No: 45.

The antibody or antigen-binding fragment thereof may comprise a FR-H1 domain of SEQ ID No: 46, which is provided herein, as follows:

[SEQ ID No: 46]
QVQLVQSGGGLVQPGGSLRLSCAAS

Thus, preferably the antibody or antigen-binding fragment thereof comprises a FR-H1 domain comprising or consisting of a sequence as substantially set out in SEQ ID No: 46, or a variant or fragment thereof.

The antibody or antigen-binding fragment thereof may comprise a FR-H2 domain of SEQ ID No: 47, which is provided herein, as follows:

[SEQ ID No: 47]
MSWVRQTPGKGLEWVSA

Thus, preferably the antibody or antigen-binding fragment thereof comprises a FR-H2 domain comprising or consisting of a sequence as substantially set out in SEQ ID No: 47, or a variant or fragment thereof.

The antibody or antigen-binding fragment thereof may comprise a FR-H3 domain of SEQ ID No: 48, which is provided herein, as follows:

[SEQ ID No: 48]
YYADSVKGRFTISRDNSKNTLYLQMNSLRAADTAVYYC

Thus, preferably the antibody or antigen-binding fragment thereof comprises a FR-H3 domain comprising or consisting of a sequence as substantially set out in SEQ ID No: 48, or a variant or fragment thereof.

The antibody or antigen-binding fragment thereof may comprise a FR-H4 domain of SEQ ID No: 49, which is provided herein, as follows:

[SEQ ID No: 49]
WGQGTLVTVSS

Thus, preferably the antibody or antigen-binding fragment thereof comprises a FR-H4 domain comprising or consisting of a sequence as substantially set out in SEQ ID No: 49, or a variant or fragment thereof.

Preferably, the antibody or antigen-binding fragment thereof comprises a FR-H1 domain comprising or consisting of SEQ ID No: 46, a FR-H2 domain comprising or consisting of SEQ ID No: 47, a FR-H3 domain comprising or consisting of SEQ ID No: 48 and/or a FR-H4 domain comprising or consisting of SEQ ID No: 49.

Preferably, the antibody or antigen-binding fragment thereof comprises a FR-H1 domain comprising or consisting of SEQ ID No: 46, a FR-H2 domain comprising or consisting of SEQ ID No: 47, a FR-H3 domain comprising or consisting of SEQ ID No: 48 and a FR-H4 domain comprising or consisting of SEQ ID No: 49.

The antibody or antigen-binding fragment thereof may comprise a heavy chain sequence as set out in SEQ ID No: 50, which is provided herein, as follows:

[SEQ ID No: 50]
QVQLVQSGGGLVQPGGSLRLSCAASGFTFSSYAMSWVRQTPGKGLEWVS
AISGSGGSTYYADSVKGRFTISRDNSKNTLYLQMNSLRAADTAVYYCAK
AGNSKLRFFDWLLTMWGQGTLVTVSS

Preferably, the antibody or antigen-binding fragment thereof comprises a heavy chain variable region comprising or consisting of a sequence as substantially set out in SEQ ID No: 50, or a variant or fragment thereof.

The antibody or antigen-binding fragment thereof may comprise a light chain CDR-L1 domain of SEQ ID No: 51, which is provided herein, as follows:

[SEQ ID No: 51]
QSVLYSSNNKNY

Thus, preferably the antibody or antigen-binding fragment thereof comprises a CDR-L1 domain comprising or consisting of a sequence as substantially set out in SEQ ID No: 51, or a variant or fragment thereof.

The antibody or antigen-binding fragment thereof may comprise a CDR-L2 domain of SEQ ID No: 52, which is provided herein, as follows:

[SEQ ID No: 52]
WAS

Thus, preferably the antibody or antigen-binding fragment thereof comprises a CDR-L2 domain comprising or consisting of a sequence as substantially set out in SEQ ID No: 52, or a variant or fragment thereof.

The antibody or antigen-binding fragment thereof may comprise a CDR-L3 domain of SEQ ID No: 53, which is provided herein, as follows:

[SEQ ID No: 53]
QQSYSSSVT

Thus, preferably the antibody or antigen-binding fragment thereof comprises a CDR-L3 domain comprising or consisting of a sequence as substantially set out in SEQ ID No: 53, or a variant or fragment thereof.

Preferably, the antibody or antigen-binding fragment thereof comprises a CDR-L1 domain comprising or consisting of SEQ ID No: 51, a CDR-L2 domain comprising or consisting of SEQ ID No: 52, and/or a CDR-L3 domain comprising or consisting of SEQ ID No: 53.

Preferably, the antibody or antigen-binding fragment thereof comprises a CDR-L1 domain comprising or consisting of SEQ ID No: 51, a CDR-L2 domain comprising or consisting of SEQ ID No: 52, and a CDR-L3 domain comprising or consisting of SEQ ID No: 53.

The antibody or antigen-binding fragment thereof may comprise a FR-L1 domain of SEQ ID No: 54, which is provided herein, as follows:

[SEQ ID No: 54]
DIQLTQSPDSLAVSLGERATINCKSS

Thus, preferably the antibody or antigen-binding fragment thereof comprises a FR-L1 domain comprising or consisting of a sequence as substantially set out in SEQ ID No: 54, or a variant or fragment thereof.

The antibody or antigen-binding fragment thereof may comprise a FR-L2 domain of SEQ ID No: 55, which is provided herein, as follows:

[SEQ ID No: 55]
LAWYQQKPGQPPKLLIY

Thus, preferably the antibody or antigen-binding fragment thereof comprises a FR-L2 domain comprising or consisting of a sequence as substantially set out in SEQ ID No: 55, or a variant or fragment thereof.

The antibody or antigen-binding fragment thereof may comprise a FR-L3 domain of SEQ ID No: 56, which is provided herein, as follows:

[SEQ ID No: 56]
TRESGVPDRFSGSGSGTDFTLTISSLQAEDVAVYYC

Thus, preferably the antibody or antigen-binding fragment thereof comprises a FR-L3 domain comprising or consisting of a sequence as substantially set out in SEQ ID No: 56, or a variant or fragment thereof.

The antibody or antigen-binding fragment thereof may comprise a FR-L4 domain of SEQ ID No: 57, which is provided herein, as follows:

[SEQ ID No: 57]
FGPGTKVEIK

Thus, preferably the antibody or antigen-binding fragment thereof comprises a FR-L4 domain comprising or consisting of a sequence as substantially set out in SEQ ID No: 57, or a variant or fragment thereof.

Preferably, the antibody or antigen-binding fragment thereof comprises a FR-L1 domain comprising or consisting of SEQ ID No: 54, a FR-L2 domain comprising or consisting of SEQ ID No: 55, a FR-L3 domain comprising or consisting of SEQ ID No: 56 and/or a FR-L4 domain comprising or consisting of SEQ ID No: 57.

Preferably, the antibody or antigen-binding fragment thereof comprises a FR-L1 domain comprising or consisting of SEQ ID No: 54, a FR-L2 domain comprising or consisting of SEQ ID No: 55, a FR-L3 domain comprising or consisting of SEQ ID No: 56 and a FR-L4 domain comprising or consisting of SEQ ID No: 57.

The antibody or antigen-binding fragment thereof may comprise a light chain sequence as set out in SEQ ID No: 58, which is provided herein, as follows:

[SEQ ID No: 58]
DIQLTQSPDSLAVSLGERATINCKSSDIQLTQSPDSLAVSLGERATINC
KSSLAWYQQKPGQPPKLLIYWASTRESGVPDRFSGSGSGTDFTLTISSL
QAEDVAVYYCQQYYSTPATFGPGTKVEIK

Preferably, the antibody or antigen-binding fragment thereof comprises a light chain variable region comprising or consisting of a sequence as substantially set out in SEQ ID No: 58, or a variant or fragment thereof.

Preferably, the antibody or antigen-binding fragment thereof comprises a CDR-H1 domain comprising or consisting of SEQ ID No: 43, a CDR-H2 domain comprising or consisting of SEQ ID No: 44, and/or a CDR-H3 domain comprising or consisting of SEQ ID No: 45, a FR-H1 domain comprising or consisting of SEQ ID No: 46, a FR-H2 domain comprising or consisting of SEQ ID No: 47, a FR-H3 domain comprising or consisting of SEQ ID No: 48 and a FR-H4 domain comprising or consisting of SEQ ID No: 49.

Preferably, the antibody or antigen-binding fragment thereof comprises a CDR-L1 domain comprising or consisting of SEQ ID No: 51, a CDR-L2 domain comprising or consisting of SEQ ID No: 52, and a CDR-L3 domain comprising or consisting of SEQ ID No: 53, a FR-L1 domain comprising or consisting of SEQ ID No: 54, a FR-L2 domain comprising or consisting of SEQ ID No: 55, a FR-L3 domain comprising or consisting of SEQ ID No: 56 and a FR-L4 domain comprising or consisting of SEQ ID No: 57.

Preferably, the antibody or antigen-binding fragment thereof comprises a CDR-H1 domain comprising or consisting of SEQ ID No: 43, a CDR-H2 domain comprising or consisting of SEQ ID No: 44, a CDR-H3 domain comprising or consisting of SEQ ID No: 45, a CDR-L1 domain comprising or consisting of SEQ ID No: 51, a CDR-L2 domain comprising or consisting of SEQ ID No: 52, and/or a CDR-L3 domain comprising or consisting of SEQ ID No: 53.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 43, a CDR-H2 domain comprising or consisting of SEQ ID No: 44, a CDR-H3 domain comprising or consisting of SEQ ID No: 45, a CDR-L1 domain comprising or consisting of SEQ ID No: 51, a CDR-L2 domain comprising or consisting of SEQ ID No: 52, and a CDR-L3 domain comprising or consisting of SEQ ID No: 53.

Preferably, the antibody or antigen-binding fragment thereof comprises a heavy chain variable region comprising or consisting of SEQ ID No: 50 and a light chain variable region comprising or consisting of SEQ ID No: 58.

### IC004

In one embodiment, the antibody or antigen-binding fragment thereof is referred to herein as IC004 (see line 4 of the tables shown in FIGS. 11A and B). The antibody or antigen-binding fragment thereof may comprise a CDR-H1 domain of SEQ ID No: 59, which is provided herein, as follows:

[SEQ ID No: 59]
GFTFSSYD

Thus, preferably the antibody or antigen-binding fragment thereof comprises a CDR-H1 domain comprising or consisting of a sequence as substantially set out in SEQ ID No: 59, or a variant or fragment thereof.

The antibody or antigen-binding fragment thereof may comprise a CDR-H2 domain of SEQ ID No: 60, which is provided herein, as follows:

[SEQ ID No: 60]
IGTAGDT

Thus, preferably the antibody or antigen-binding fragment thereof comprises a CDR-H2 domain comprising or consisting of a sequence as substantially set out in SEQ ID No: 60, or a variant or fragment thereof.

The antibody or antigen-binding fragment thereof may comprise a CDR-H3 domain of SEQ ID No: 61, which is provided herein, as follows:

[SEQ ID No: 61]
GGPSVWLLLLLLRYGR

Thus, preferably the antibody or antigen-binding fragment thereof comprises a CDR-H3 domain comprising or consisting of a sequence as substantially set out in SEQ ID No: 61, or a variant or fragment thereof.

The inventors surprisingly discovered that the nucleotide sequence encoding the CDR-H3 domain of the antibody of IC004 (SEQ ID No: 61) comprises a stop codon between the sequences encoding Tryptophan (W) and Leucine (L), and as indicated by * in the table of FIG. 11A. Despite the presence of the stop codon in the open reading frame, the inventors were very surprised to be able to produce a full length antibody comprising the full length CDR-H3 sequence as shown in SEQ ID No: 61. This is especially significant given that the CDH-H3 provides great potential for diversity for antigen binding, and is important in conferring binding activity and specificity.

Preferably, the antibody or antigen-binding fragment thereof comprises a CDR-H1 domain comprising or consisting of SEQ ID No: 59, a CDR-H2 domain comprising or consisting of SEQ ID No: 60, and/or a CDR-H3 domain comprising or consisting of SEQ ID No: 61.

Preferably, the antibody or antigen-binding fragment thereof comprises a CDR-H1 domain comprising or consisting of SEQ ID No: 59, a CDR-H2 domain comprising or consisting of SEQ ID No: 60, and a CDR-H3 domain comprising or consisting of SEQ ID No: 61.

The antibody or antigen-binding fragment thereof may comprise a FR-H1 domain of SEQ ID No: 62, which is provided herein, as follows:

[SEQ ID No: 62]
VQLVESGGGLVQPGGSLRLSCAAS

Thus, preferably the antibody or antigen-binding fragment thereof comprises a FR-H1 domain comprising or consisting of a sequence as substantially set out in SEQ ID No: 62, or a variant or fragment thereof.

The antibody or antigen-binding fragment thereof may comprise a FR-H2 domain of SEQ ID No: 63, which is provided herein, as follows:

[SEQ ID No: 63]
MHWVRQATGKGLEWVSA

Thus, preferably the antibody or antigen-binding fragment thereof comprises a FR-H2 domain comprising or consisting of a sequence as substantially set out in SEQ ID No: 63, or a variant or fragment thereof.

The antibody or antigen-binding fragment thereof may comprise a FR-H3 domain of SEQ ID No: 64, which is provided herein, as follows:

[SEQ ID No: 64]
YYPGSVKGRFTISRENAKNSLYLQMNSLRAGDTAVYYF

Thus, preferably the antibody or antigen-binding fragment thereof comprises a FR-H3 domain comprising or consisting of a sequence as substantially set out in SEQ ID No: 64, or a variant or fragment thereof.

The antibody or antigen-binding fragment thereof may comprise a FR-H4 domain of SEQ ID No: 65, which is provided herein, as follows:

[SEQ ID No: 65]
LGPRDHGHRLL

Thus, preferably the antibody or antigen-binding fragment thereof comprises a FR-H4 domain comprising or consisting of a sequence as substantially set out in SEQ ID No: 65, or a variant or fragment thereof.

Preferably, the antibody or antigen-binding fragment thereof comprises a FR-H1 domain comprising or consisting of SEQ ID No: 62, a FR-H2 domain comprising or consisting of SEQ ID No: 63, a FR-H3 domain comprising or consisting of SEQ ID No: 64 and/or a FR-H4 domain comprising or consisting of SEQ ID No: 65.

Preferably, the antibody or antigen-binding fragment thereof comprises a FR-H1 domain comprising or consisting of SEQ ID No: 62, a FR-H2 domain comprising or consisting of SEQ ID No: 63, a FR-H3 domain comprising or consisting of SEQ ID No: 64 and a FR-H4 domain comprising or consisting of SEQ ID No: 65.

The antibody or antigen-binding fragment thereof may comprise a heavy chain sequence as set out in SEQ ID No: 66, which is provided herein, as follows:

[SEQ ID No: 66]
EVQLVESGGGLVQPGGSLRLSCAASGFTFSSYDMHWVRQATGKGLEWVS
AIGTAGDTYYPGSVKGRFTISRENAKNSLYLQMNSLRAGDTAVYYFGGP
SVWLLLLLLRYGR

Preferably, the antibody or antigen-binding fragment thereof comprises a heavy chain variable region comprising or consisting of a sequence as substantially set out in SEQ ID No: 66, or a variant or fragment thereof.

The antibody or antigen-binding fragment thereof may comprise a light chain CDR-L1 domain of SEQ ID No: 67, which is provided herein, as follows:

[SEQ ID No: 67]
SSNIGAGYD

Thus, preferably the antibody or antigen-binding fragment thereof comprises a CDR-L1 domain comprising or consisting of a sequence as substantially set out in SEQ ID No: 67, or a variant or fragment thereof.

The antibody or antigen-binding fragment thereof may comprise a CDR-L2 domain of SEQ ID No: 68, which is provided herein, as follows:

[SEQ ID No: 68]
GNS

Thus, preferably the antibody or antigen-binding fragment thereof comprises a CDR-L2 domain comprising or consisting of a sequence as substantially set out in SEQ ID No: 68, or a variant or fragment thereof.

The antibody or antigen-binding fragment thereof may comprise a CDR-L3 domain of SEQ ID No: 69, which is provided herein, as follows:

[SEQ ID No: 69]
QSYDSSLSGSV

Thus, preferably the antibody or antigen-binding fragment thereof comprises a CDR-L3 domain comprising or consisting of a sequence as substantially set out in SEQ ID No: 69, or a variant or fragment thereof.

Preferably, the antibody or antigen-binding fragment thereof comprises a CDR-L1 domain comprising or consisting of SEQ ID No: 67, a CDR-L2 domain comprising or consisting of SEQ ID No: 68, and/or a CDR-L3 domain comprising or consisting of SEQ ID No: 69.

Preferably, the antibody or antigen-binding fragment thereof comprises a CDR-L1 domain comprising or consisting of SEQ ID No: 67, a CDR-L2 domain comprising or consisting of SEQ ID No: 68, and a CDR-L3 domain comprising or consisting of SEQ ID No: 69.

The antibody or antigen-binding fragment thereof may comprise a FR-L1 domain of SEQ ID No: 70, which is provided herein, as follows:

[SEQ ID No: 70]
QSVLTQPPSVSGAPGQRVTISCTGS

Thus, preferably the antibody or antigen-binding fragment thereof comprises a FR-L1 domain comprising or consisting of a sequence as substantially set out in SEQ ID No: 70, or a variant or fragment thereof.

The antibody or antigen-binding fragment thereof may comprise a FR-L2 domain of SEQ ID No: 71, which is provided herein, as follows:

[SEQ ID No: 71]
VHWYQQLPGTAPKLLIY

Thus, preferably the antibody or antigen-binding fragment thereof comprises a FR-L2 domain comprising or consisting of a sequence as substantially set out in SEQ ID No: 71, or a variant or fragment thereof.

The antibody or antigen-binding fragment thereof may comprise a FR-L3 domain of SEQ ID No: 72, which is provided herein, as follows:

[SEQ ID No: 72]
NRPSGVPDRFSGSKSGTSASLAITGLQAEDEADYYC

Thus, preferably the antibody or antigen-binding fragment thereof comprises a FR-L3 domain comprising or consisting of a sequence as substantially set out in SEQ ID No: 72, or a variant or fragment thereof.

The antibody or antigen-binding fragment thereof may comprise a FR-L4 domain of SEQ ID No: 73, which is provided herein, as follows:

[SEQ ID No: 73]
FGGGTKLTVL

Thus, preferably the antibody or antigen-binding fragment thereof comprises a FR-L4 domain comprising or consisting of a sequence as substantially set out in SEQ ID No: 73, or a variant or fragment thereof.

Preferably, the antibody or antigen-binding fragment thereof comprises a FR-L1 domain comprising or consisting of SEQ ID No: 70, a FR-L2 domain comprising or consisting of SEQ ID No: 71, a FR-L3 domain comprising or consisting of SEQ ID No: 72 and/or a FR-L4 domain comprising or consisting of SEQ ID No: 73.

Preferably, the antibody or antigen-binding fragment thereof comprises a FR-L1 domain comprising or consisting of SEQ ID No: 70, a FR-L2 domain comprising or consisting of SEQ ID No: 71, a FR-L3 domain comprising or consisting of SEQ ID No: 72 and a FR-L4 domain comprising or consisting of SEQ ID No: 73.

The antibody or antigen-binding fragment thereof may comprise a light chain sequence as set out in SEQ ID No: 74, which is provided herein, as follows:

[SEQ ID No: 74]
QSVLTQPPSVSGAPGQRVTISCTGSSSNIGAGYDVHWYQQLPGTAPKLL
IYGNSNRPSGVPDRFSGSKSGTSASLAITGLQAEDEADYYCQSYDSSLS
GSVFGGGTKLTVL

Preferably, the antibody or antigen-binding fragment thereof comprises a light chain variable region comprising or consisting of a sequence as substantially set out in SEQ ID No: 74, or a variant or fragment thereof.

Preferably, the antibody or antigen-binding fragment thereof comprises a CDR-H1 domain comprising or consisting of SEQ ID No: 59, a CDR-H2 domain comprising or consisting of SEQ ID No: 60, and a CDR-H3 domain comprising or consisting of SEQ ID No: 61, a FR-H1 domain comprising or consisting of SEQ ID No: 62, a FR-H2 domain comprising or consisting of SEQ ID No: 63, a FR-H3 domain comprising or consisting of SEQ ID No: 64 and a FR-H4 domain comprising or consisting of SEQ ID No: 65.

Preferably, the antibody or antigen-binding fragment thereof comprises a CDR-L1 domain comprising or consisting of SEQ ID No: 67, a CDR-L2 domain comprising or consisting of SEQ ID No: 68, and a CDR-L3 domain comprising or consisting of SEQ ID No: 69, a FR-L1 domain comprising or consisting of SEQ ID No: 70, a FR-L2 domain comprising or consisting of SEQ ID No: 71, a FR-L3 domain comprising or consisting of SEQ ID No: 72 and a FR-L4 domain comprising or consisting of SEQ ID No: 73.

Preferably, the antibody or antigen-binding fragment thereof comprises a CDR-H1 domain comprising or consisting of SEQ ID No: 59, a CDR-H2 domain comprising or consisting of SEQ ID No: 60, a CDR-H3 domain comprising or consisting of SEQ ID No: 61, a CDR-L1 domain comprising or consisting of SEQ ID No: 67, a CDR-L2 domain comprising or consisting of SEQ ID No: 68, and/or a CDR-L3 domain comprising or consisting of SEQ ID No: 69.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 59, a CDR-H2 domain comprising or consisting of SEQ ID No: 60, a CDR-H3 domain comprising or consisting of SEQ ID No: 61, a CDR-L1 domain comprising or consisting of SEQ ID No: 67, a CDR-L2 domain comprising or consisting of SEQ ID No: 68, and a CDR-L3 domain comprising or consisting of SEQ ID No: 69.

Preferably, the antibody or antigen-binding fragment thereof comprises a heavy chain variable region comprising or consisting of SEQ ID No: 66 and a light chain variable region comprising or consisting of SEQ ID No: 74.

Preferably, the antibody or antigen-binding fragment thereof may comprise any combination of CDR and/or FR sequences disclosed herein.

In addition, as described in Isaacs et al. (1998); J. Immun.; 161:3862-3869 (“Therapy with monoclonal antibodies—The contribution of the Fc gamma receptor binding and the influence of CH1 and CH3 domains on in vivo effector function”, the antibody or antigen binding fragment of the invention may comprise one or more mutation within a motif critical for FcγR binding. For example, the mutations may comprise a glutamate 233 to proline, a leucine/phenylalanine 234 to valine, and/or a leucine 235 to alanine substitution.

Advantageously, the antibody or antigen-binding fragment thereof according to the first aspect of the invention has utility as a therapeutic agent in its own right, and may be used in the treatment, amelioration or prevention of coronavirus infection.

Accordingly, in a second aspect of the invention, there is provided an antibody or an antigen-binding fragment thereof according to the first aspect, for use in therapy.

In a third aspect of the invention, there is provided an antibody or an antigen-binding fragment thereof according to the first, for use in treating, preventing or ameliorating coronavirus infection.

According to a fourth aspect of the invention, there is provided a method of treating, preventing or ameliorating coronavirus infection in a subject, the method comprising administering, or having administered, to a patient in need of such treatment, a therapeutically effective amount of an antibody or antigen-binding fragment thereof according to the first aspect.

Preferably, the coronavirus infection is an infection of a coronavirus as defined in the first aspect.

The antibody or antigen binding g fragment described herein may provide an effective means of vaccinating a subject against coronavirus infection.

Accordingly, in a fifth aspect of the invention, there is provided a vaccine comprising an antibody or antigen-binding fragment thereof according to the first aspect.

Preferably, the vaccine comprises a suitable adjuvant. The adjuvant may be an encoded molecular adjuvant or as adjuvant incorporated into a delivery formulation.

The adjuvant incorporated into a delivery formulation may be selected form the group consisting of a bacterial lipopeptide, lipoprotein and lipoteichoic acid; mycobacterial lipoglycan; yeast zymosan, porin, Lipopolysaccharide, Lipid A, monophosphoryl lipid A (MPL), Flagellin, CpG DNA, hemozoin, Saponins (Quil-A, QS-21, Tomatine, ISCOM, ISCOMATRIX™), squalene based emulsions, polymers such as PEI, Carbopol, lipid nanoparticles and bacterial toxins (CT, LT).

In a sixth aspect of the invention, there is provided an antibody or an antigen-binding fragment thereof according to the first aspect or a vaccine according to the fifth aspect, for use in stimulating an immune response in a subject.

The immune response may be stimulated against coronavirus, preferably SARS-CoV, MERS-CoV and/or SARS CoV2 infection.

It will be appreciated that antibodies, fragments thereof or a vaccine according to the invention (collectively referred to herein as “agents”) may be used in a monotherapy (e.g. the use of an antibody or antigen binding fragment thereof alone), for treating, ameliorating or preventing coronavirus infection. Alternatively, agents according to the invention may be used as an adjunct to, or in combination with, known therapies for treating, ameliorating, or preventing coronavirus infection.

The agents according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising agents of the invention may be used in a number of ways. For instance, oral administration may be required, in which case the agents may be contained within a composition that may, for example, be ingested orally in the form of a tablet, capsule or liquid. Compositions comprising agents and medicaments of the invention may be administered by inhalation (e.g. intranasally). Compositions may also be formulated for topical use. For instance, creams or ointments may be applied to the skin.

Agents and medicaments according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with agents used according to the invention is required and which would normally require frequent administration (e.g. at least daily injection).

In a preferred embodiment, agents and medicaments according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the antibodies and fragments (i.e. agent) that is required is determined by its biological activity and bioavailability, which in turn depends on the mode of administration, the physiochemical properties of the agent, and whether it is being used as a monotherapy or in a combined therapy. The frequency of administration will also be influenced by the half-life of the agent within the subject being treated. Optimal dosages to be administered may be determined by those skilled in the art, and will vary with the particular agent in use, the strength of the pharmaceutical composition, the mode of administration, and the advancement of the coronavirus infection. Additional factors depending on the particular subject being treated will result in a need to adjust dosages, including subject age, weight, gender, diet, and time of administration.

Generally, a daily dose of between 0.001 μg/kg of body weight and 10 mg/kg of body weight of agent according to the invention may be used for treating, ameliorating, or preventing coronavirus infection, depending upon which agent. More preferably, the daily dose of agent is between 0.01 μg/kg of body weight and 1 mg/kg of body weight, more preferably between 0.1 μg/kg and 100 μg/kg body weight, and most preferably between approximately 0.1 μg/kg and 10 μg/kg body weight.

The agent may be administered before, during or after onset of coronavirus infection. Daily doses may be given as a single administration (e.g. a single daily injection). Alternatively, the agent may require administration twice or more times during a day. As an example, agents may be administered as two (or more depending upon the severity of the coronavirus infection being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of agents according to the invention to a patient without the need to administer repeated doses. Known procedures, such as those conventionally employed by the pharmaceutical industry (e.g. in vivo experimentation, clinical trials, etc.), may be used to form specific formulations of the agents according to the invention and precise therapeutic regimes (such as daily doses of the agents and the frequency of administration).

In an seventh aspect of the invention, there is provided a pharmaceutical composition comprising an antibody or antigen-binding fragment thereof according to the first aspect, or a vaccine according to the fifth aspect, and optionally a pharmaceutically acceptable vehicle.

The pharmaceutical composition is preferably an anti-coronavirus composition, i.e. a pharmaceutical formulation used in the therapeutic amelioration, prevention or treatment of coronavirus infection in a subject, such as a SARS-CoV, MERS-CoV and/or SARS CoV2 infection.

The invention also provides in a eighth aspect, a process for making the pharmaceutical composition according to the fourth aspect, the process comprising combining a therapeutically effective amount of an antibody or antigen-binding fragment thereof as defined in the first aspect, or a vaccine according to the fifth aspect, with a pharmaceutically acceptable vehicle.

The antibody or antigen-binding fragment thereof may be as defined with respect to the first aspect.

A “subject” may be a vertebrate, mammal, or domestic animal. Hence, medicaments according to the invention may be used to treat any mammal, for example livestock (e.g. a horse), pets, or may be used in other veterinary applications. Most preferably, the subject is a human being.

A “therapeutically effective amount” of the antibody or antigen-binding fragment thereof is any amount which, when administered to a subject, is the amount of agent that is needed to treat the coronavirus infection, or produce the desired effect.

For example, the therapeutically effective amount of antibody or fragment thereof used may be from about 0.001 ng to about 1 mg, and preferably from about 0.01 ng to about 100 ng. It is preferred that the amount of antibody or fragment is an amount from about 0.1 ng to about 10 ng, and most preferably from about 0.5 ng to about 5 ng.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

As discussed herein, the antibodies of the invention are able to bind to the spike protein S2 domain of SARS-CoV, MERS-CoV and SARS CoV2, which acts as the epitope. This finding suggests that the antibodies or antigen-binding fragments thereof may be used as a robust diagnostic tool for detecting coronavirus infection.

Thus, according to a ninth aspect, there is provided a method for diagnosing a coronavirus infection in a subject, or prognosing a subject's condition, the method comprising detecting the presence of coronavirus in a sample obtained from a subject, wherein detection is achieved using an antibody or antigen binding fragment according to the first aspect.

According to a tenth aspect there is provided the antibody or antibody binding fragment of according to the first aspect, for use in diagnosis or prognosis.

According to an eleventh aspect of the invention, there is provided the antibody or antibody binding fragment of according to the first aspect, for use in diagnosing or prognosing coronavirus infection.

According to the twelfth aspect of the invention, there is provided a kit for diagnosing a subject suffering from coronavirus infection, or for providing a prognosis of the subject's condition, the kit comprising an antibody or antigen-binding fragment thereof according to the first aspect for detecting coronavirus in a sample from a test subject.

Preferably, the coronavirus in relation to any of the ninth to twelfth aspect is as defined in the first aspect.

Prognosis may relate to determining the therapeutic outcome in a subject that has been diagnosed with coronavirus infection. Prognosis may relate to predicting the rate of progression or improvement and/or the duration of coronavirus infection in a subject, the probability of survival, and/or the efficacy of various treatment regimes. Thus, a poor prognosis may be indicative of coronavirus infection progression, low probability of survival and reduced efficacy of a treatment regime. A favourable prognosis may be indicative of coronavirus infection resolution, high probability of survival and increased efficacy of a treatment regime.

Preferably, the sample comprises a biological sample. The sample may be any material that is obtainable from a subject from which protein is obtainable.

The biological sample may be tissue or a biological fluid. The biological sample may be any material that is obtainable from the subject from which monocytes are obtainable. Furthermore, the sample may be blood, plasma, serum, spinal fluid, urine, sweat, saliva, tears, breast aspirate, breast milk, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, lymph, granuloma, cancer biopsy or combinations thereof.

The sample may be a liquid aspirate. For example, the sample may be bronchial alveolar lavage (BAL), ascites, pleural lavage, or pericardial lavage.

The sample may comprise blood, urine, tissue etc. In one preferred embodiment, the biological sample comprises a blood sample. The blood may be venous or arterial blood. Blood samples may be assayed immediately. Alternatively, the blood sample may be stored at low temperatures, for example in a fridge or even frozen before the method is conducted. Alternatively, the blood sample may be stored at room temperature, for example between 18 to 22 degrees Celsius, before the method is conducted. The blood sample may comprise comprises blood serum. The blood sample may comprise blood plasma. Preferably, however the detection is carried out on whole blood and most preferably the blood sample is peripheral blood.

The blood may be further processed before the use of the first aspect is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting.

The invention also extends to the use of the above-identified epitopes of coronavirus as antigens for the production of the antibodies or antibody fragments of the invention.

Accordingly, in a thirteenth aspect of the invention, there is provided the use of the spike protein S2 domain, or region of the spike protein domain, as defined in the first aspect, as an antigen.

Preferably, the antigen acts as an epitope to which antibody binds.

Preferably, the antigen comprises or consists of a sequence as substantially set out in SEQ ID No: 6, or a variant or fragment thereof.

In a fourteenth aspect, there is provided an antibody or antigen-binding fragment thereof obtained by a method comprising:—
(i) immunising a host organism with the spike protein S2 domain, or a region of the spike protein domain, as defined in the first aspect; and
(ii) collecting an antibody or antigen-binding fragment thereof from the host.

The host may be a mammal, and may be a human, rabbit or mouse.

Preferably, the method comprises bleeding the host animal, and then preferably collecting the antibody or antigen-binding fragment thereof from the blood, most preferably blood serum. Preferably, the blood serum is passed through a gravity column with covalently bound peptide-support. Following washing, the antibody or antigen-binding fragment thereof is preferably eluted in buffer, which is preferably acidic buffer, and the solution may then be neutralized. The method may further comprise dialysis against a suitable buffer (e.g. PBS) and, optionally, lyophilisation.

In a fifteen aspect of the invention, there is provided a polynucleotide sequence encoding the antibody or antigen binding fragment thereof as defined in the first aspect.

In a sixteenth aspect of the invention, there is provided an expression cassette comprising a polynucleotide sequence according to the fifteenth aspect.

The polynucleotide sequence encoding the antibody or antigen binding fragment thereof of the invention is preferably harboured in a recombinant vector, for example a recombinant vector for delivery into a host cell of interest to enable production of the antibody or antigen binding fragment thereof.

Accordingly, in a seventeenth aspect of the invention there is provided a recombinant vector comprising the expression cassette according to the sixteenth aspect.

The vector encoding the antibody or antigen binding fragment thereof of the first aspect may for example be a plasmid, cosmid or phage and/or be a viral vector. Such recombinant vectors are highly useful in the delivery systems of the invention for transforming cells with the nucleotide sequences. The nucleotide sequences may preferably be a DNA sequence, and it is this DNA sequence which encodes the antibody or antigen binding fragment thereof sequence forming the antibody or antigen binding fragment thereof of the second aspect.

Recombinant vectors encoding the antibody or antigen binding fragment thereof of the first aspect may also include other functional elements. For example, they may further comprise a variety of other functional elements including a suitable promoter for initiating transgene expression upon introduction of the vector in a host cell. For instance, the vector is preferably capable of autonomously replicating in the nucleus of the host cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, EF1a, PGK, viral long terminal repeats, as well as inducible promoters, such as the Tetracycline inducible system, as examples. The cassette or vector may also comprise a terminator, such as the Beta globin, SV40 polyadenylation sequences or synthetic polyadenylation sequences. The recombinant vector may also comprise a promoter or regulator or enhancer to control expression of the nucleic acid as required.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the transgene. The cassette or vector may also comprise DNA involved with regulating expression of the nucleotide sequence, or for targeting the expressed polypeptide to a certain part of the host cell.

Purified vector may be inserted directly into a host cell by suitable means, e.g. direct endocytotic uptake. The vector may be introduced directly into a host cell (e.g. a eukaryotic or prokaryotic cell) by transfection, infection, electroporation, microinjection, cell fusion, protoplast fusion, calcium phosphate, cationic lipid-based lipofection, polymer or dendrimer-based methods or ballistic bombardment.

Alternatively, vectors of the invention may be introduced directly into a host cell using a particle gun.

Alternatively, the delivery system may provide the polynucleotide to the host cell without it being incorporated in a vector. For instance, the nucleic acid molecule may be incorporated within a liposome or virus particle. Alternatively a “naked” polynucleotide may be inserted into a host cell by a suitable means e.g. direct endocytotic uptake.

In an eighteenth aspect of the invention, there is provided a host cell comprising the polynucleotide sequence according to the fifteenth aspect, the expression cassette according to the sixteenth aspect, or the vector according to the seventeen aspect.

The host cell may be a eukaryotic or prokaryotic host cell. Preferably, the host cell is a eukaryotic host cell. More preferably, the host cell is a mammalian host cell such as NS0 murine myeloma cells, PER.C6® human cells, Human embryonic kidney 293 cells or Chinese hamster ovary (CHO) cells. Most preferably, the host cell is a CHO cell.

In a nineteenth aspect, there is provided a method of preparing the antibody or antigen binding fragment according to the first aspect, the method comprising:
a) introducing, into a host cell, the vector of the seventeenth aspect; and
b) culturing the host cell under conditions to result in the production of the antibody or antigen binding fragment according to the first aspect.

The host cell of step a) may be a eukaryotic or prokaryotic host cell. Preferably, the host cell is a eukaryotic host cell. More preferably, the host cell is a mammalian host cell such as NS0 murine myeloma cells, PER.C6® human cells, Human embryonic kidney 293 cells or Chinese hamster ovary (CHO) cells. Most preferably, the host cell is a CHO cell.

The method may further comprise (c) harvesting, centrifuging and/or filtering the cell culture media to obtain a cell culture supernatant comprising the antibody or antigen binding fragment thereof.

The method may further comprise (d) separating and purifying the antibody or antigen binding fragment thereof from the cell culture supernatant. Preferably, purification is performed by at least one chromatographic step.

Suitable chromatographic steps include affinity chromatography and/or ion exchange chromatography. Preferably, affinity chromatography is protein A chromatography. Ion exchange chromatography may be anionic exchange chromatography and/or cationic exchange chromatography.

Preferably, step (d) comprises separating and purifying the antibody or antigen binding fragment thereof from the cell culture supernatant by:
i) protein A chromatography;
ii) anionic exchange chromatography; and/or
iii) cationic exchange chromatography.

The method may further comprise (e) filtering the purified antibody or antigen binding fragment thereof resulting from step (d). Preferably, step (e) comprises virus filtration. Thus, preferably the purified antibody or antigen binding fragment thereof resulting from step (d) is filtered using a virus filtration membrane. Suitable membranes would be known to those skilled in the art.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-89 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on: —(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula: −Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, in those of SEQ ID Nos: 1 to 89 that are amino acid sequences.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

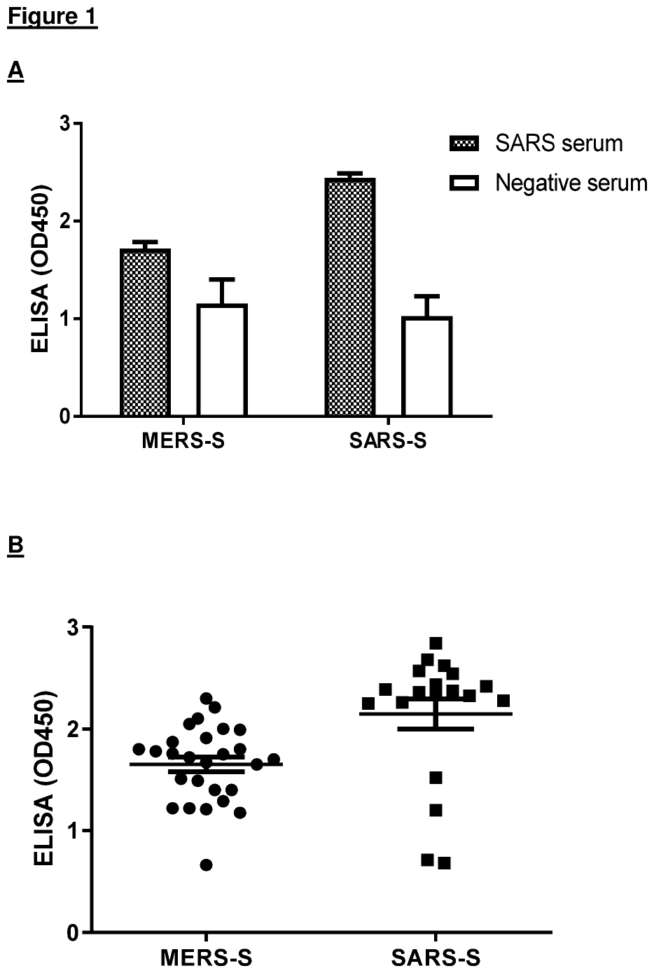

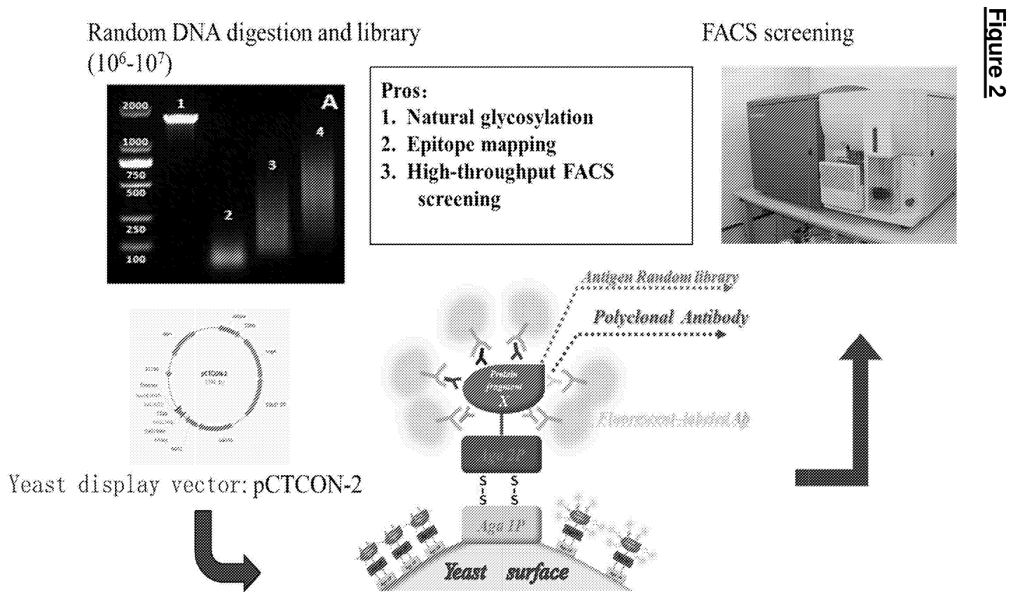

FIG. 1 shows the results of an ELISA for MERS specific IgG when compared with SARS specific IgG in SARS convalescent sera. Sera from a cohort of SARS convalescence show their binding to the spike protein of SARS-CoV vs MERS-CoV by ELISA indicating the serological cross-reactivity between the two viruses.

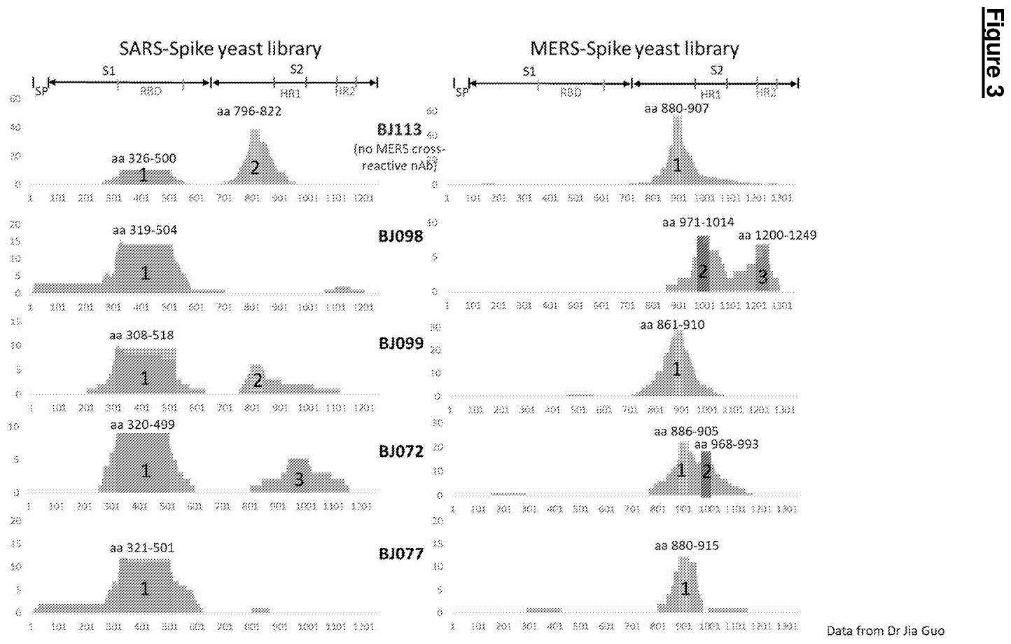

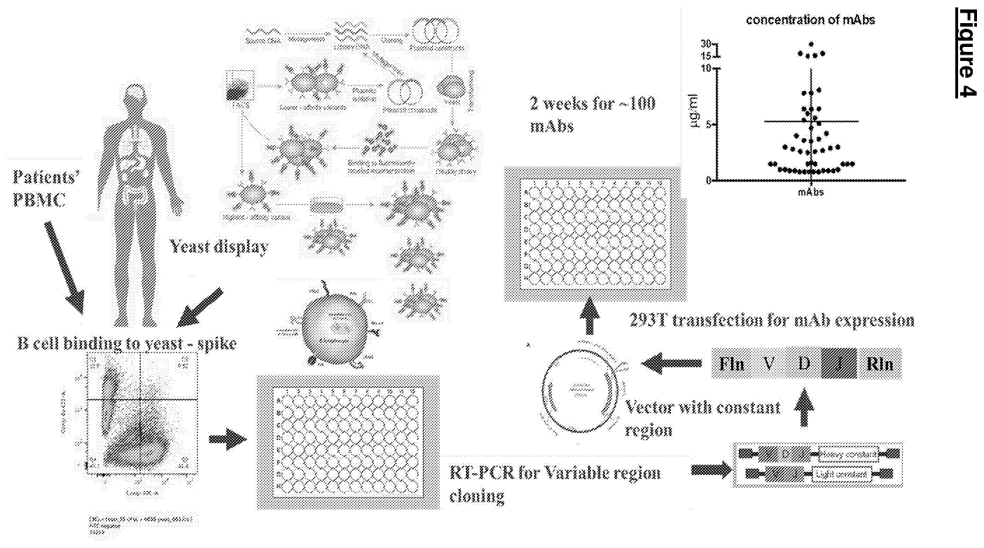

FIG. 2 shows the Yeast Surface display antigen library (YSDL) platform for serum antibody profiling.

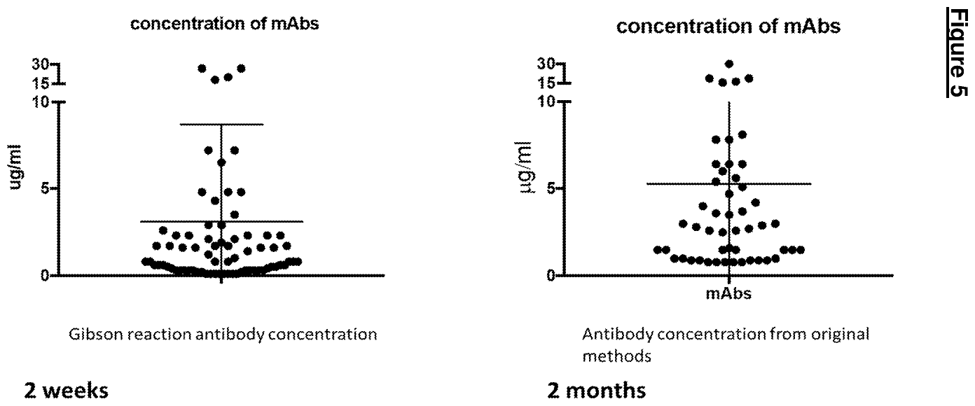

FIG. 3 shows the SARS-Spike yeast library and the MERS-Spike yeast library. The YSDL of Spike protein from SARS and MERS was used to screen SARS convalescent sera, and cross-reactive antibody epitopes in the S2 domain of S protein were identified.

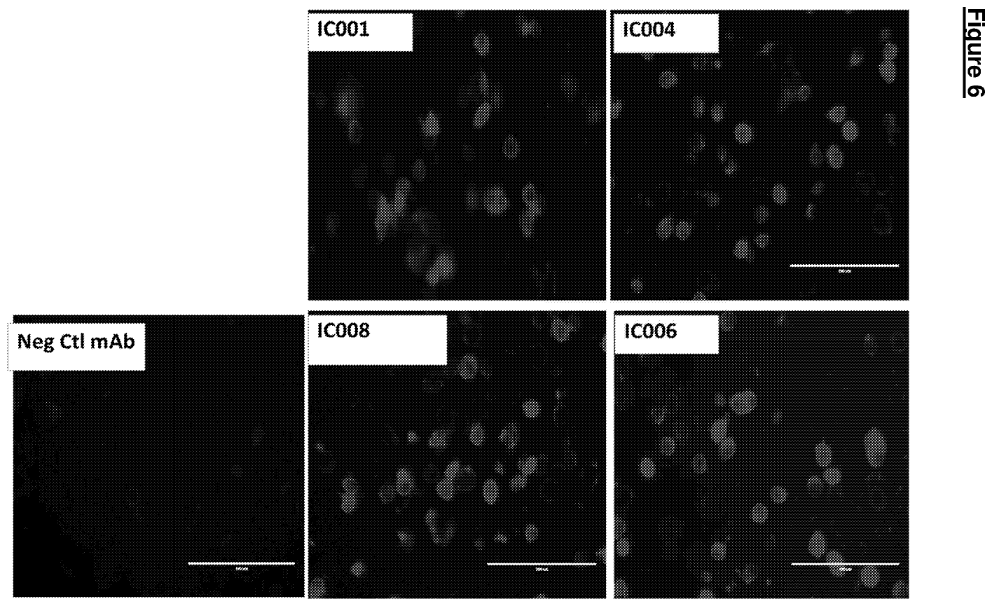

FIG. 4 shows the high-throughput human mAb cloning strategy use by the inventors using YSDL.

FIG. 5 shows new monoclonal antibody cloning methods, which indicates that the strategy summarised in FIG. 4 is more efficient in terms of time required, reducing the development time to 2 weeks when compared to 2 months of standard protocols.

FIG. 6 shows the binding specificity of the top 4 CoV-bnMAB to the membrane form of S protein of SARS-CoV-2 expressed in 293T cells.

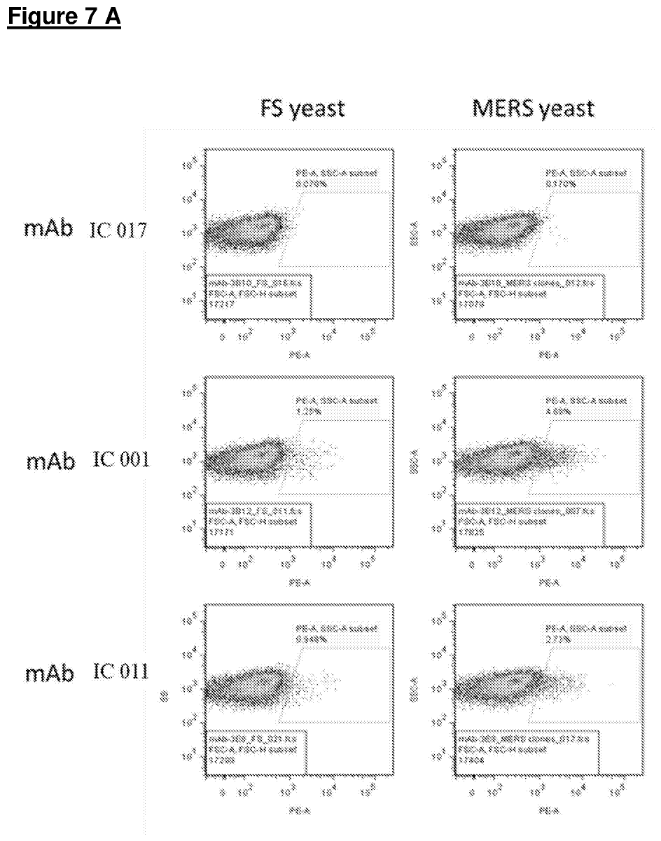

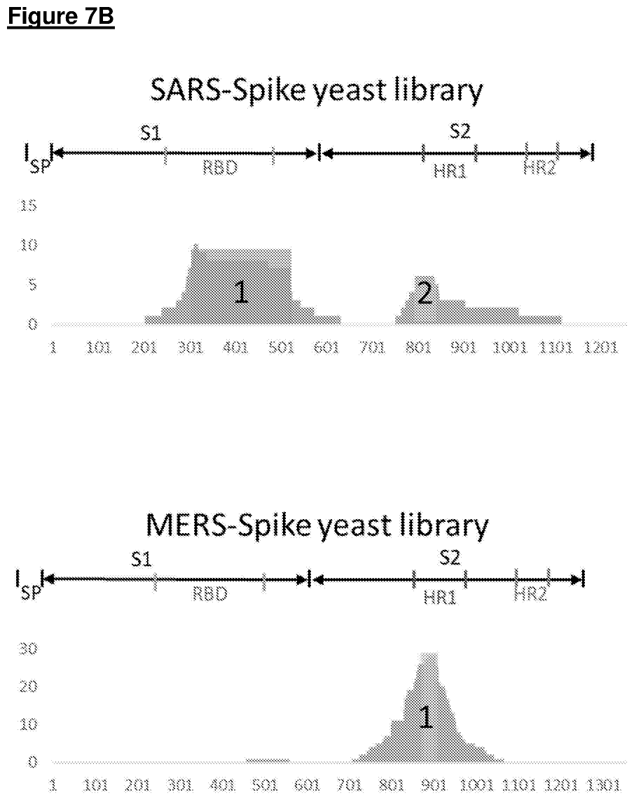

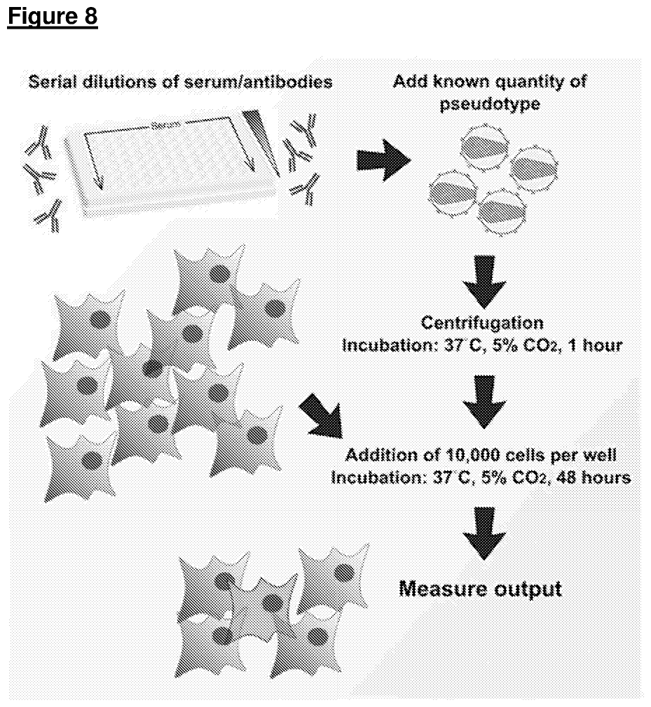

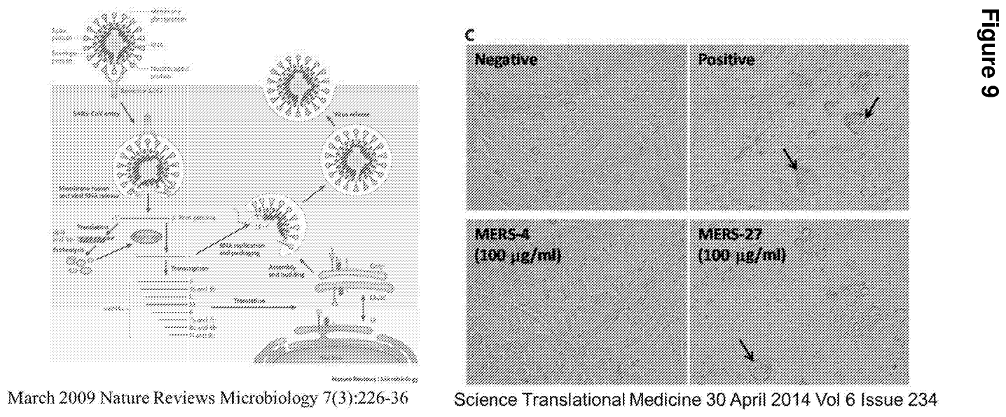

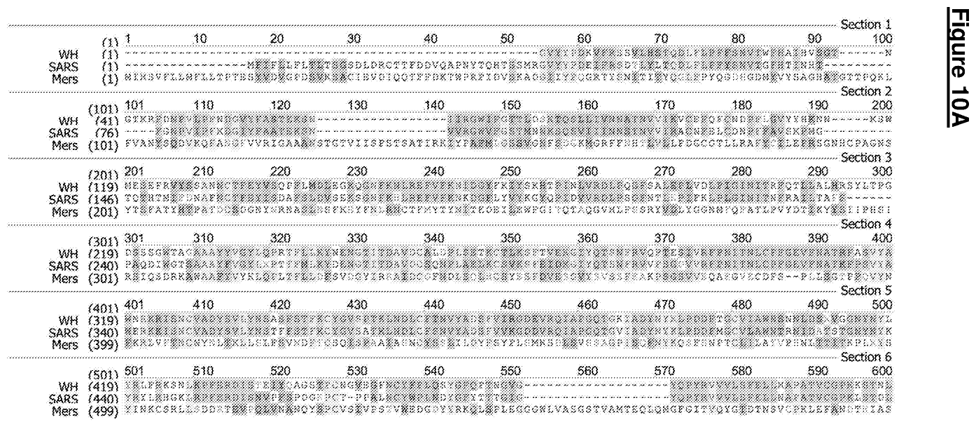

FIG. 7 shows that cross-reactive anti-MERS CoV S mAbs bind to yeast-spike 2.

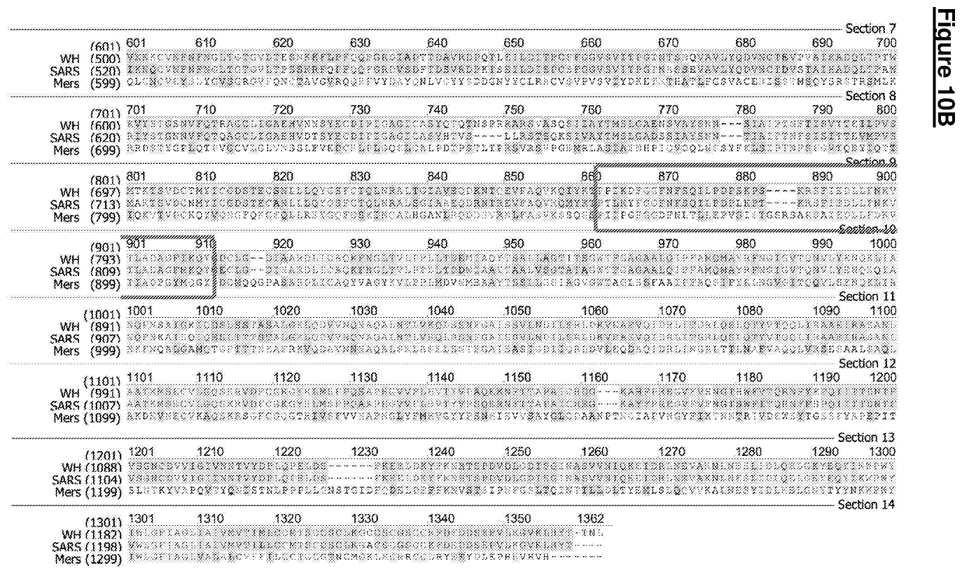

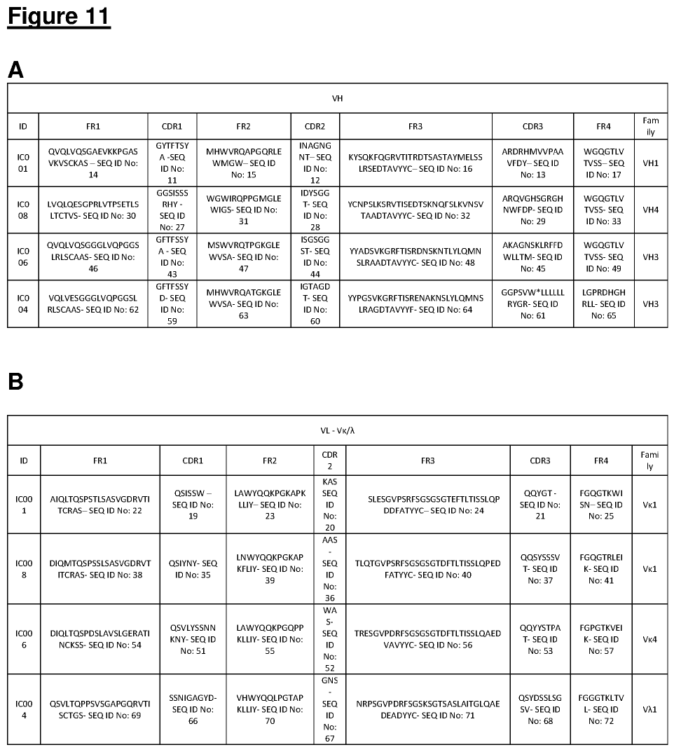

FIG. 8 shows that the monoclonal antibodies of the invention neutralize pseudotype MERS-CoV.

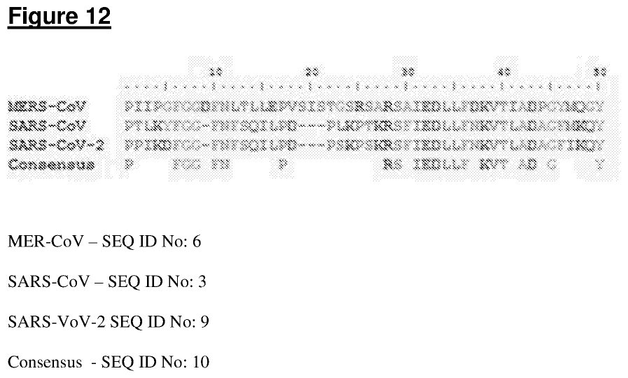

FIG. 9 shows previously published data showing that SARS-CoV and MERS-CoV fuse to the membrane of target cells.

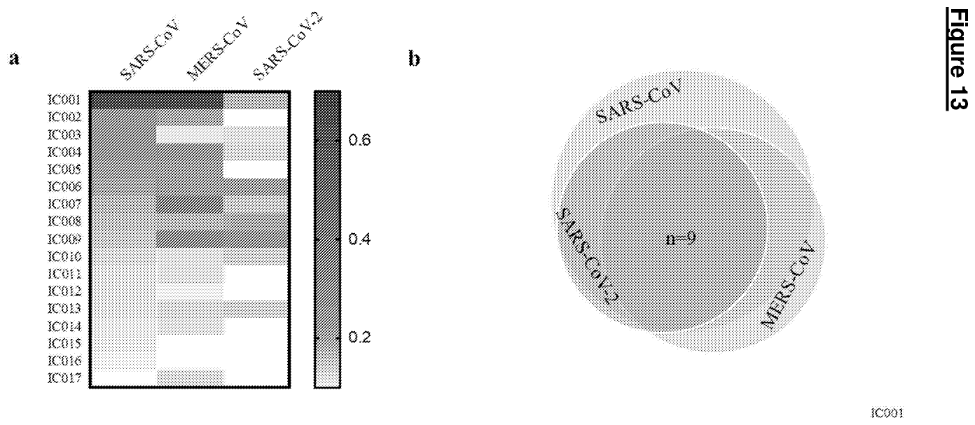

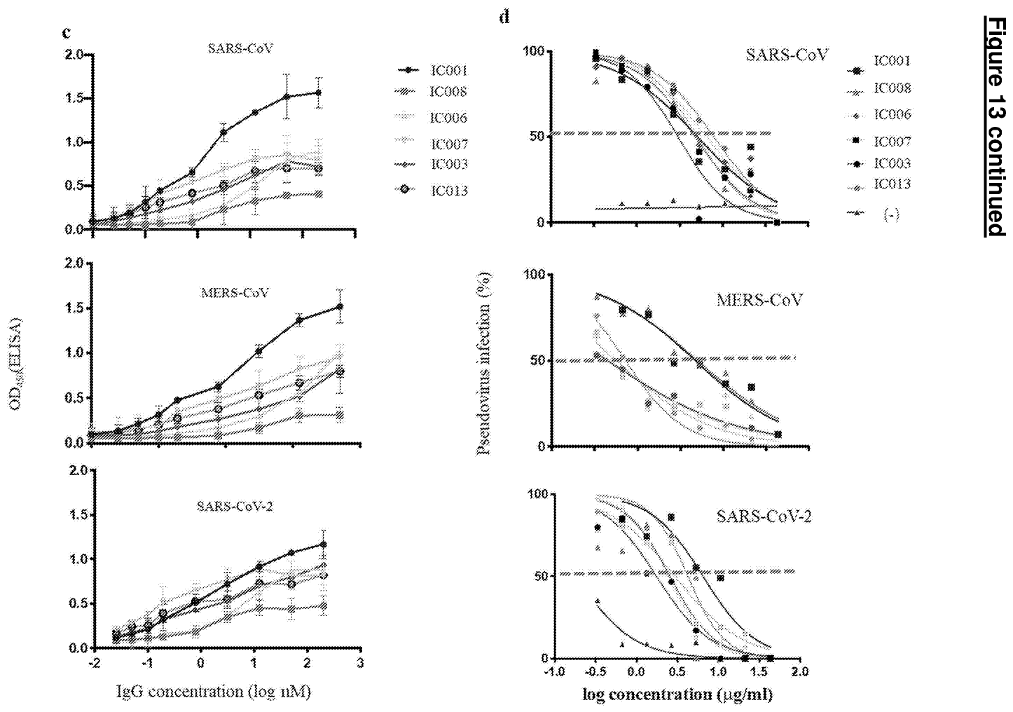

FIGS. 10A and B shows a sequence alignment between SARS-CoV, MERS-CoV and SARS-CoV2 spike proteins.

FIGS. 11A and B shows the heavy chain and light chain sequences of four of the antibodies that the inventors has found to be surprisingly cross-reactive against SARS-CoV, MERS-CoV and SARS-CoV2.

FIG. 12 shows the sequences of the cross-reactive domain of S2 region in MERS-CoV, SARS-CoV and SARS-CoV-2.

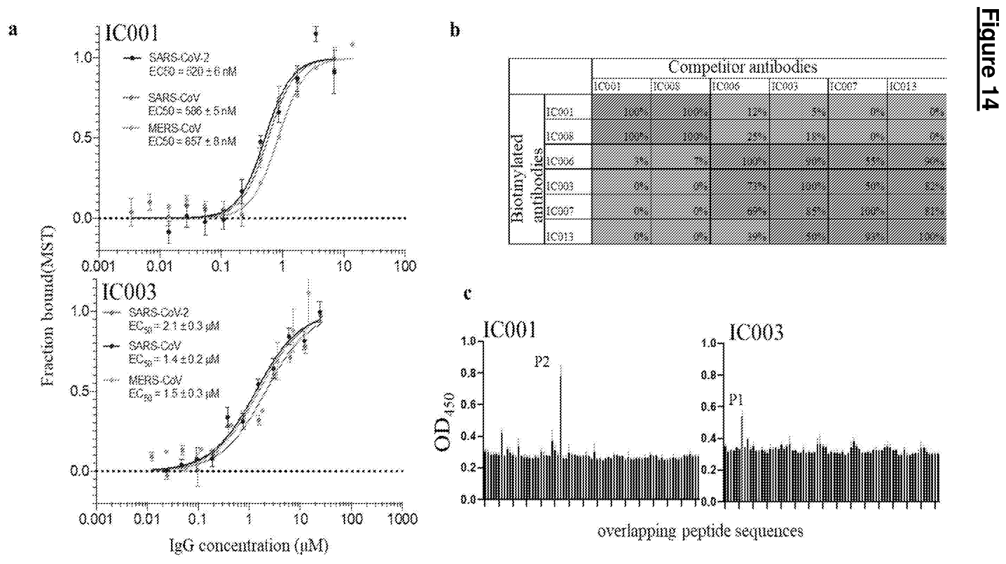

FIG. 13 shows that the monoclonal antibodies developed by the inventors bind and neutralize SARS-CoV, MERS-CoV and SARS-CoV-2. A). Heat map summarizing the seventeen mAbs binding to SARS-CoV, MERS-CoV and SARS-CoV-2 at 1 μg/ml by ELISA. B). pie chart describing the number of mAbs that bind to all three coronavirus. N=9. C). Dose dependent binding of mAbs to SARS-CoV, MERS-CoV and SARS-CoV-2 spike protein by ELISA. Experiment was repeated three times. Mean and SD is calculated. D). neutralization potency of the mAbs by pseudovirus SARS-CoV, MERS-CoV and SARS-CoV-2. Percentage of pseudovrirus inhibition is plotted. Data is representative of two independent study. The dash line indicate the 50% reduction in viral infectivity (IC50). IC50 is calculated by non-linear regression. E) Neutralization potency of mAbs by cytopathic effect (CPE) with authentic SARS-CoV, MERS-CoV and SARS-CoV-2. Vero cell is infected with the virus and the protection is scored by comparing with non-infected control. Representative figures of the infected cells treated with and without mAbs are shown. F) Inhibition activity of mAbs against SARS-CoV, MERS-CoV and SARS-CoV-2 mediated cell fusion at 100 μg/ml. Representative figures of the mAbs co-incubated with spike transfected 293T cells with ACE2/DPP4 expressing Huh7 cells were shown.

FIG. 14 shows the binding affinity of the antibodies and epitope mapping of the mAbs. A). Microscale thermophoresis (MST)-binding curves using purified mAbs versus fluorescently labeled SARS-CoV, MERS-CoV and SARS-CoV spike trimer. Normalized fluorescence was calculated using NanoTemper Analysis 1.2.101 and is plotted as function of mAb concentration. The effective half maximal binding (EC50) was calculated by non-linear regression of the hill equation in Prism 8.4.3. N=3 B). Antibody epitopes were defined by cross-inhibition for binding. The CR3022 was used as a guide mAb to distinguish anti-RBD antibodies. The influenza and ebola antibodies were used as non-inhibiting controls. The mAbs cloned from the patients were used to test the inhibition effect of each other by ELISA as described in methods below. Red designates inhibition of the biotinylated antibody to the competing antibody while green designates the absence of inhibition. N=3 C). The selected mAbs were screened against a library of overlapping peptides that covers the SARS-CoV-2 spike. D). The peptides that were recognized are represented on the structure of SARS-CoV spike monomer. PDB: 6VXX, E). Shows embodiments of P1 and P2 epitopes in the spike protein S2 domain in each of SARS-CoV, MERS-CoV and SARS-CoV.

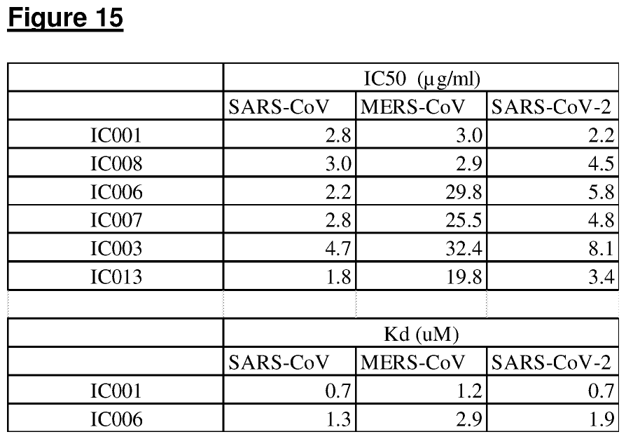

FIG. 15 shows a table summarising the neutralization potential of the antibodies.

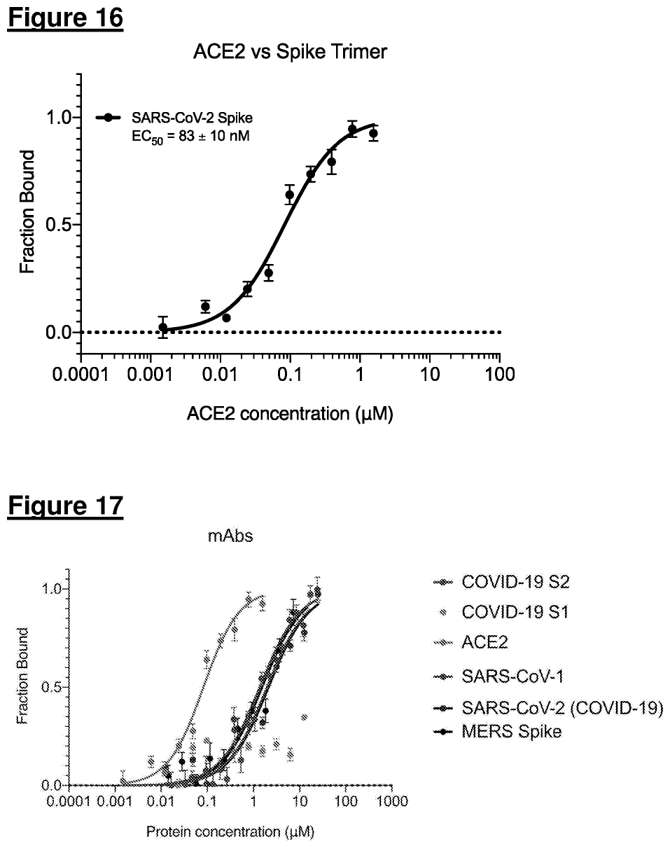

FIG. 16 shows the binding affinity of the ACE2 to the spike as a reference.

FIG. 17 shows binding of the mAbs to S1 and S2 region of the spike. As expected, all the mAbs recognize the S2 region of the SARS-CoV-2 but not the S1 region.

FIG. 18 shows the percentage neutralisation of the antibodies IC001, IC005, IC009, IC010, IC013, IC008, IC004, IC03 and IC006 against various mutant or variant coronavirus, including (i) two bat coronavirus variants that are similar to SARS-CoV, i.e. WIV16 and RaTG13; (ii) SARS-CoV-2 mink mutant; (iii) SARS-CoV-2 B1.1.7 variant (Kent mutant); and (iv) SARS-CoV-2 B.1.351 variant (South Africa variant).

### EXAMPLES

The inventors initially set out to produce antibodies with cross-reactivity between coronavirus SARS-CoV and MERS-CoV. Due to new highly pathogenic coronavirus (2019-nCoV renamed as SARS-CoV-2 by WHO on 11 Feb. 2020) outbreak which occurred in December 2019 in China, the inventors extended their work to produce antibodies that were cross-reactive with multiple coronavirus strains, including SARS-CoV, MERS-CoV and SARS-CoV-2. Sequence alignments found that the S protein (the major target of antibody response) of SARS-CoV-2 shares higher homology with SARS-CoV (77%) than compared to MERS-CoV (35%).

The inventors hypothesized that some of CoV monoclonal antibodies generated from SARS patients could cross-react with SARS-CoV-2 in addition to MERS-CoV and SARS-CoV, and the data that the inventors have obtained show that the CoV monoclonal antibodies can specifically bind to SARS-CoV-2 S protein expressed on the surface of 293T cells, as well as MERS-CoV and SARS-CoV S protein. Thus, the inventors believe that they are the first to develop antibodies that display cross-reactivity to SARS-CoV-2, MERS-CoV and SARS-CoV S, providing a pan coronavirus antibody that may be used for detecting and treating all known and future coronavirus infections.

### Materials and Methods

Constructing YSD Spike library for SARS-CoV, MERS-CoV and SARS CoV2 DNA sequences encoding SARS-CoV, MERS-CoV and SARS Cova spike were amplified by PCR using the Expand High Fidelity PCR System (Cat. No. 11732650001, Roche). The PCR products were then purified, digested and ligated to pCTCON2 vector. The ligation products were transformed into competent TG1 cells. After sequencing, recombinant plasmids with 100% homology were prepared and termed as pCTCON2-SARS-CoV, MERS-CoV and SARS CoV2 accordingly. Competent EBY100 cells were then transformed with pCTCON2-SARS-CoV, MERS-CoV and SARS CoV2 via EP 0.1 mm gene pulser cuvette (Cat. No. 165-2089, Bio-Rad) using Gene Pulser Xcell™ Total System (Cat. No. 165-2660, Bio-Rad) with EP parameters of 1.2 kv, 25 uF and resistance 2000. Then, yeast cells were cultured for 1 h at 30° C. followed by spreading on SDCAA plates and culture for 48 h at 30° C. Single colonies were picked from the selective plates and cultured in SDCAA media, followed by induction of surface fragments expression in SGCAA media. Surface expression of SARS-CoV, MERS-CoV and SARS CoV2 spike fragments was verified by analysis of surface expression of c-Myc tag with flow cytometry. For epitope mapping, after being cultured in SGCAA media for 30 h at 4° C., fragment-expressing yeast cells were analyzed by flow cytometry with SARS patients' sera at various dilutions.

Sera profiling by VSD libraries (FACS sorting and yeast sequencing) Sera profiling was carried out based on the yeast surface display (YSD) library as previously described (Zuo et al. 2011; Guo et al. 2015). Briefly, the combinatorial fragment libraries of SARS-CoV, MERS-CoV and SARS-CoV-2 spike was constructed and displayed on the surface of yeast respectively for antibody staining and Fluorescence-activated cell sorting (FACS). Specifically, the full-length Spike gene was digested and PCR-reassembled into a range of 100-1000 bp fragments, the reassembled fragments were gel purified and cloned into yeast surface display vector. The cloned products were then transformed into competent yeast cell line EBY100 using electroporation.

The yeast library was induced and incubated with individual or pooled SARS serum and positive sorted by FACS using Aria III (BD, USA). The sorted positive yeast clones displaying the respective antigenic fragments were harvested and the plasmids encoding the corresponding fragments were extracted and subjected to sequencing and sequence analysis.

### Single Memory B Cell Isolation by Yeast Clones and mAb Production

Monoclonal antibodies were isolated from single B cells that bound by yeast-spike. PBMCs from SARS patients were thawed with cold Foetal Bovine Sera at 4° C. and then co-cultured with BV421 labelled MERS spike yeast at 1:2 ratio. The PBMC MERS spike yeast mixture were then washed and the CD19+BV421t cells were single cell sorted into 96 well plates containing 10 μl RNase-inhibiting RT-PCR catch buffer (5 ml RNase-free water, 50 μl 1M Tris pH8 and 125 μl RNasin (Promega). Plates were immediately sealed and frozen on dry ice and stored at −80° C. Single cell cDNA was synthesized in the original sort plates by adding 15 μl RT-PCR reaction mix. RT-PCR reaction mix contains 1 μl forward primer mix (1.2 μM), 1 μl reverse primer mix (1.2 μM) 1 μl dNTPs (200 μM), 5 μl 10*buffer, 0.5 μl PCR enzyme mix and 6.5 μl H₂O. Individual IgH and IgL(k or λ) genes were amplified in the 2nd round PCR reaction with HotstarTaq PCR kit. IgH and Ig λ genes are digested with AgeI/SalI or AgeI/XhoI respectively prior to the ligation into the variable gene cloning site of the IgH/IgL expression vectors. The expression vectors are composed of the appropriate human constant region downstream of a murine immunoglobulin signal peptide and ampicillin resistant gene. The Igk products were sequenced and amplified according to its gene family by another round of PCR. They were then digested with AgeI/BsiWI before ligated into Igk expression vector. IgH and IgL chain containing plasmids were mixed with PEI (Polyethylenimine) and transfected into human embryonic kidney fibroblast 293T cells. Cells were washed with DMEM/PBS 24 hr after transfection and then cultured in protein free media Supernatants are collected five-six days after transfection.

### Antibody Purification

293T cells transfected with VH and VL plasmids were cultured in PF Ultradoma (Lonza) for 5 days before supernatant were collected, filtered (0.2 μm) and treated with MNase (2.5 U/ml) for 120 min. The supernatant is then loaded to column packed with protein A beads, and washed with PBS three times before eluted with glycine pH 2.7 and neutralized with Tris-HCl pH 8. The purified antibody is subsequently buffer-exchanged with PBS and stored in −80° C.

### ELISA Binding

NUNC ELISA plates were coated with anti-His (abcam) antibody at 1.5 μg/ml overnight at 4° C. The plates were then washed with PBST and blocked with 3% BSA (Sigma) for 1 hour at room temperature. SARS-CoV, MERS-CoV and SARS-CoV2 spike trimer was added to the plates at the same concentration as anti-His antibody. Afterwards, mAb or patients serum was added to the plates in various dilutions. The plates were washed twice before anti-human IgG alkaline phosphatase (Sigma) addition. After PBST washing, PNPP (p-Nitrophenyl phosphate) solution (Sigma) was used to develop colour for OD reading.

### In Vitro Neutralization Assays

Neutralizing activity of mAbs against the SARS CoV, MERS CoV and SARS CoV2 strains was analysed in a microneutralization (MN) assay. Vero-E6 cell were seeded at 3×10\hat{ }4 cell per well on 10×96 well plate in DMEM+10% FBS+1% P/S. Monoclonal antibody was serial diluted in quadruplicate column in 96 well U-bottom plate (three for CPE score, one for no virus control NVC/cytotoxic control). 60 μl plain DMEM was added to row B to H and 60 μl diluted antibody was added to row A and B. mAbs were serial diluted with multi-channel pipette by transfer 60 ul mixed antibody from row B to C down row H.

60 ul diluted virus were to three columns of diluted antibody and 60 ul plain DMEM was added to NVC column with one hour incubation at 37° C. 100 ul of antibody-virus mix was then transferred to culture plate with Vero-E6 cell from row H to row A. Each well contain 100 TCID50 virus and antibody concentration start from 10, 5, 2.5, 1.25, 0.625. 0.313, 0.156 and 0.078 μg/ml. The vero cell, mAb and virus mixture was incubated at 5% CO2, 37° C. incubator for 72 hours. Score the cytopathic effect (CPE) in record sheet and determine the neutralization antibody concentration against 100× TCID 50 virus. Neutralization titre reported as Full protection from CPE cause by 100× TCID50 virus inoculum with reference to the No Virus Control (NVC).

### In Vitro Neutralization Assays

Epitope mapping of the mAbs was carried out based on the yeast surface display (YSD) library. The combinatorial fragment library of SARS-CoV, MERS-CoV and SARS CoV2 spike was constructed and displayed on the surface of yeast for antibody staining and Fluorescence-activated cell sorting (FACS). Specifically, the full-length spike gene was digested and PCR-reassembled into a range of 100-900 bp fragments, the reassembled fragments were gel purified and cloned into yeast surface display vector. The cloned products were then transformed into competent yeast cell line EBY100 using electroporation. The yeast library was induced and incubated with each of the anti-MERS/SARS CoV/SARS CoV2 mAbs and positive sorted by FACS using Aria III (BD, USA). The sorted positive yeast clones displaying the respective antigenic fragments were harvested and the plasmids encoding the corresponding fragments were extracted and subjected to sequencing and sequence analysis.

Protein expression and purification SARS-CoV and MERS-CoV S ectodomain protein was kindly given by G.Gao*. To express the COVID19 S ectodomain, a gene encoding residue 1-1208 of COVID19 Spike (GenBank: YP009724390.1) a C-terminal T4 fibritin trimerization motif, an HRV3C protease cleavage site, a StrepTag was synthesized and cloned into the insect expression vector pFBDM. The expression vector was transformed into Bacmids using Tn7 transposition methods. Transfection and virus amplification were conducted with Sf9 cells, and His cells (Invitrogen) were used to produce the recombinant proteins. Soluble S protein was captured from cell supernatants by StrepTrap HP 5 ml column (GE Healthcare). The eluted product was pooled and further purified by gel filtration chromatography with a Superose 6 10/300 GL (GE Healthcare) column equilibrated with a buffer containing 20 mM Tris-HCl (pH8.0), 150 mM NaCl and 1 mM EDTA.

### Results & Discussion

Referring to FIG. 1, there are shown the sera from a cohort of SARS convalescence showing the binding to the spike protein of SARS-CoV vs MERS-CoV by ELISA indicating that there is serological cross-reactivity between the two viruses.

The inventors utilised a Yeast Surface Display Antigen Library (YSDL) platform for serum antibody profiling, as shown in FIG. 2. Using the YSDL of Spike protein from SARS and MERS, the inventors were able to screen SARS convalescent sera, to identify cross-reactive antibody epitopes. In particular, the S2 domain of S protein was identified, as shown in FIG. 3. The inventors utilised a high-throughput monoclonal antibodies (mAbs) screening strategy, as shown in FIG. 4, using YSDL. This strategy proved to be much more efficient in terms of time required to produce the antibodies, reducing the time to 2 weeks, compared to 2 months based on standard protocols (FIG. 5).

The inventors then tested the four surprisingly effective CoV-bnMABs for their binding specificity to the membrane form of S protein of SARS-CoV-2 expressed in 293T cells. The four antibody sequences are provided in FIGS. 11A and B. As shown in FIG. 6, the antibodies were able to bind to the S protein. The inventors also confirmed the ability of the cross-reactive anti-MERS CoV S mAbs to bind to yeast-spike 2 protein, as shown in FIG. 7.

The inventors cloned 270 mAbs in total that displayed unique VDJ sequences using their system. They screened the mAbs against SARS-CoV, SARS-CoV-2 and MERS-CoV spikes by enzyme-linked-immunosorbent assay (ELISA) (FIG. 13A), 9 of them bound to all three spikes.

The binding curves displayed comparable avidity of mAbs to SARS-CoV, MERS-CoV spike and SARS-CoV-2 spike with micro- to nanomolar affinity (FIG. 13C). In particular, IC001 was particularly effective and bound to SARS-CoV and MERS-CoV to nanomolar affinity. The inventors then tested the neutralization of the mAbs against SARS-CoV, MERS-CoV and SARS-CoV-2 pseudovirus. Contrary to the published results, the inventors found that anti-S2 mAbs also neutralized SARS-CoV-2 pseudovirus (IC50: 2.2 to 8.1 μg/ml) (FIG. 18D). The mAbs showed the best neutralization effect against SARS-CoV (IC50: 1.8 to 4.7 μg/ml) among the three coronavirus. IC001 showed the strongest neutralization ability among the mAbs for both SARS-CoV(1.8 μg/ml) and MERS-CoV (2.9 μg/ml), which is correlated with its high binding avidity to the spike proteins of the three coronavirus (FIG. 13C).

Furthermore, IC001 also showed neutralization against mutant SARS-CoV-2 pseudovirus, including SARS-CoV-2 mink mutant, SARS-CoV-2 B1.1.7 (Kent mutant) and two bat coronavirus that are similar to SARS-CoV, i.e. RaTG13 and WIV16. Interestingly, IC006 showed neutralization to SARS-CoV-2, B1.1.17 and B.1.351 (South Africa) mutants, but not to the bat coronavirus, which might indicate the epitope is not conserved in bat coronavirus (FIG. 18).

To evaluate the antibodies neutralization against authentic live virus, the inventors performed the cytopathic effect (CPE) inhibition assay with mAbs on live SARS-CoV, MERS-CoV and SARS-CoV-2. IC001 showed complete neutralization against all three coronavirus (5 μg/ml for SARS-CoV and MERS-CoV, 10 μg/ml for SARS-CoV-2). IC006 showed complete neutralization against SARS-CoV and SARS-CoV-2 at 10 μg/ml (FIG. 13E). The neutralization potential of the IC001 and IC006 is correlated with their binding avidity to the three coronavirus spike (FIG. 15). To further study the neutralization mechanism of the mAbs, the inventors tested the fusion inhibition ability of the mAbs. IC001 displayed 60% and 80% fusion inhibition against SARS-CoV-2 and MERS-CoV respectively, consistent with its neutralization ability. IC008 on the other hand, displayed strong fusion inhibition against SARS-CoV-2 but not against MERS-15 CoV, further confirms its neutralization potency (FIG. 13F).

The specific interaction between the mAbs and three coronavirus spike protein was measured by microscale thermophoresis (MST) assay. The MST data show that the IC001 interact with SARS-CoV and SARS-CoV-2 spike with similar affinity of 520 and 20) 586 nM respectively. It bound to MERS-CoV spike with affinity of 857 nM (FIG. 14A). On the contrary, IC006 bound to SARS-CoV and MERS-CoV spike with similar affinity (1.4 and 1.5 μM respectively) but a lot weaker to the SARS-CoV-2 (2.1 μM). The inventors also measured the binding affinity of the ACE2 to the spike as a reference (83 nM) (FIG. 16). Although the binding affinity of IC001 is around 7 times weaker than 25 the ACE2, the neutralization ability of IC001 is still comparable to mAbs with affinity 10 folds higher than ACE2. The inventors then tried to define the epitopes of the mAbs with three assays. First, they measured the mAbs binding to S1 and S2 region of the spike. As expected, all the mAbs recognize the S2 region of the SARS-CoV-2 but not the S1 (FIG. 17). Second, the inventors measured the competitive inhibition of binding of mAbs to each other. They found that IC001 and IC008 shared similar epitopes while 30 IC006, IC003, IC007 and IC013 shared similar epitopes (FIG. 14B). Third, the inventors quantified the binding of the mAbs to overlapping peptides that cover the S2 region. IC001 bound strongly to the HR1 region while IC006 bound to FP region (FIG. 14C). The inventors have been able to identify two shared epitope regions in the 35 S2 region based on these data, highlighted in FIG. 14D as P1 and P2.

### CONCLUSIONS

The coronavirus family has been identified recently in a number of emerging pathogen priority lists i.e. UKVN, WHO blueprint and CEPI, highlighting the urgent need to improve our understanding of immune response to coronaviruses both to control current problems and to be prepared for emerging threats. The large number of genetically distinct CoV and increasing interface between human populations and animal reservoirs of CoV suggests that there is a significant risk of new CoV zoonotic infections in humans. Indeed, the outbreak of SARS-CoV in 2003, MERS-CoV in 2012, and the recent SARS-CoV-2 in 2019, has proved this to be the case. Currently there is no effective treatment or vaccine available for CoV infections in humans, largely due to the diversity of CoV family in wildlife and periodic zoonotic transmission from animal hosts to humans.

The inventors have demonstrated a novel epitope on the S2 region of the three betacoronaviruses identified by yeast display system. To their best knowledge, they are the first to produce mAbs that recognize and bind to the spike of SARS-CoV, SARS-CoV-2 and MERS-CoV. These mAbs advantageously neutralize all three betacoronaviruses and enable pan-coronavirus neutralization.

Therefore, the inventor's development of human CoV broadly neutralizing mAbs (CoV-bnMAB)-based immunotherapies for the highly pathogenic CoVs will address an immediate unmet medical need and could prove a rapid treatment not only for current known human CoVs but also for future unknown emerging pandemic CoVs.

In addition, in view of the high mortality associated with pandemic human CoV infections in particular in the elderly, it remains a matter of great importance to develop effective broad-spectrum antibodies such as the antibodies described herein. These antibodies will not only for the treatment of infected patients but also for diagnosis of infections, and also the prophylactic protection for the first-line health care personnel who are at risk and can be stockpiled against future outbreaks.

1. An antibody or antigen-binding fragment thereof that binds to the spike protein S2 domain of severe acute respiratory syndrome coronavirus (SARS-CoV), Middle East respiratory syndrome-related coronavirus (MERS-CoV) and/or severe acute respiratory syndrome coronavirus 2 (SARS CoV-2).

2. The antibody or antigen-binding fragment thereof according to claim 1, wherein the antibody or antigen-binding fragment thereof is cross-reactive with SARS-CoV, MERS-CoV and SARS CoV2.

3. The antibody or antigen-binding fragment thereof according to claim 1, wherein the antibody or antigen-binding fragment thereof neutralises SARS-CoV, MERS-CoV and SARS CoV2.

4. The antibody or antigen-binding fragment thereof according to claim 1, wherein the antibody or antigen-binding fragment thereof:
(i) binds to the spike protein S2 domain of SARS-CoV and MERS-CoV;
(ii) binds to the spike protein S2 domain of SARS-CoV and SARS CoV2;
(iii) binds to the spike protein S2 domain of MERS-CoV and SARS CoV2; and/or
(iv) binds to the spike protein S2 domain of SARS-CoV, MERS-CoV and SARS CoV2.

5. The antibody or antigen-binding fragment thereof according to claim 1, wherein the antibody or antigen-binding fragment thereof binds to between amino acid positions 711 and 728 and/or 878 and 898 of the SARS-CoV spike protein substantially as set out in SEQ ID No:1.

6. The antibody or antigen-binding fragment thereof according to claim 1, wherein the antibody or antigen-binding fragment thereof binds to a region between amino acid positions 797 and 814 and/or 970 and 990 of the MERS-CoV spike protein substantially as set out in SEQ ID No:4.

7. The antibody or antigen-binding fragment thereof according to claim 1, wherein the antibody or antigen-binding fragment thereof binds to a region between amino acid positions 729 and 746 and/or 896 and 916 of the SARS-CoV2 spike protein substantially as set out in SEQ ID No:7.

8. The antibody or antigen-binding fragment thereof according to claim 1, wherein the antibody or antigen-binding fragment thereof binds to a sequence comprising or consisting of a sequence selected from: SEQ ID No: 80, SEQ ID No: 81 and SEQ ID No: 82 or variants or fragments thereof.

9. The antibody or antigen-binding fragment thereof according to claim 1, wherein the antibody or antigen-binding fragment thereof binds to a sequence comprising or consisting of a sequence selected from: SEQ ID No: 76, SEQ ID No: 77 and SEQ ID No: 78, or variants or fragments thereof.

10. The antibody or antigen-binding fragment thereof according to claim 1, wherein the antibody or antigen-binding fragment thereof binds to a sequence comprising or consisting of a sequence as substantially set out in SEQ ID No: 83.

11. The antibody or antigen-binding fragment thereof according to claim 1, wherein the antibody or antigen-binding fragment thereof binds to a sequence comprising or consisting of a sequence as substantially set out in SEQ ID No: 79.

12. The antibody or antigen-binding fragment thereof according to claim 1, wherein the antibody or antigen-binding fragment thereof comprises a CDR-H1 domain comprising SEQ ID No: 11, a CDR-H2 domain comprising SEQ ID No: 12, a CDR-H3 domain comprising SEQ ID No: 13, a CDR-L1 domain comprising SEQ ID No: 19, a CDR-L2 domain comprising SEQ ID No: 20, and/or a CDR-L3 domain comprising SEQ ID No: 21, optionally wherein the antibody or antigen-binding fragment thereof comprises at least one, at least two, at least three, at least four, at least five, or at least six of the CDRs.

13. The antibody or antigen-binding fragment thereof according to claim 1, wherein the antibody or antigen-binding fragment thereof comprises a CDR-H1 domain comprising SEQ ID No: 27, a CDR-H2 domain comprising SEQ ID No: 28, a CDR-H3 domain comprising SEQ ID No: 29, a CDR-L1 domain comprising SEQ ID No: 35, a CDR-L2 domain comprising SEQ ID No: 36, and/or a CDR-L3 domain comprising SEQ ID No: 37, optionally wherein the antibody or antigen-binding fragment thereof comprises at least one, at least two, at least three, at least four, at least five, or at least six of the CDRs.

14. The antibody or antigen-binding fragment thereof according to claim 1, wherein the antibody or antigen-binding fragment thereof comprises a CDR-H1 domain comprising SEQ ID No: 43, a CDR-H2 domain comprising SEQ ID No: 44, a CDR-H3 domain comprising SEQ ID No: 45, a CDR-L1 domain SEQ ID No: 51, a CDR-L2 domain comprising SEQ ID No: 52, and/or a CDR-L3 domain comprising SEQ ID No: 53, optionally wherein the antibody or antigen-binding fragment thereof comprises at least one, at least two, at least three, at least four, at least five, or at least six of the CDRs.

15. The antibody or antigen-binding fragment thereof according to claim 1, wherein the antibody or antigen-binding fragment thereof comprises a CDR-H1 domain comprising SEQ ID No: 59, a CDR-H2 domain SEQ ID No: 60, a CDR-H3 domain comprising SEQ ID No: 61, a CDR-L1 domain comprising SEQ ID No: 67, a CDR-L2 domain comprising SEQ ID No: 68, and/or a CDR-L3 domain comprising SEQ ID No: 69, optionally wherein the antibody or antigen-binding fragment thereof comprises at least one, at least two, at least three, at least four, at least five, or at least six of the CDRs.

16. (canceled)

17. A method of treating, preventing or ameliorating coronavirus infection, the method comprising administering an antibody or an antigen-binding fragment thereof that binds to the spike protein S2 domain of severe acute respiratory syndrome coronavirus (SARS-CoV), Middle East respiratory syndrome-related coronavirus (MERS-CoV) and/or severe acute respiratory syndrome coronavirus 2 (SARS CoV-2).

18. (canceled)

19. A vaccine comprising an antibody or antigen-binding fragment thereof according to claim 1 or a pharmaceutical composition comprising an antibody or antigen-binding fragment thereof according to claim 1 and, optionally wherein the vaccine or pharmaceutical composition further comprises an adjuvant and/or a pharmaceutically acceptable vehicle.

20-23. (canceled)

24. A method for diagnosis or prognosis of coronavirus infection in a subject, the method comprising detecting the presence the disease in a sample obtained from a subject, wherein detection is achieved using an antibody or antibody binding fragment thereof that binds to the spike protein S2 domain of severe acute respiratory syndrome coronavirus (SARS-CoV), Middle East respiratory syndrome-related coronavirus (MERS-CoV) and/or severe acute respiratory syndrome coronavirus 2 (SARS CoV-2).

25. (canceled)

26. A kit for diagnosing a subject suffering from coronavirus infection, or for providing a prognosis of the subject's condition, the kit comprising an antibody or antigen-binding fragment thereof according to claim 1 for detecting coronavirus in a sample from a test subject.

27-33. (canceled)


SEQUENCE LISTING (89 sequences)

SEQ ID NO: 1 - protein, 1255 aa, SARS-CoV
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 595 aa, SARS-CoV
ASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKL

SEQ ID NO: 3 - protein, 46 aa, SARS-CoV
PTLKYFGGFNFSQILPDPLKPTKRSFIEDLLFNKVTLADAGFMKQY

SEQ ID NO: 4 - protein, 1353 aa, MERS-CoV
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSHTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 5 - protein, 582 aa, MERS-CoV
VDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVH

SEQ ID NO: 6 - protein, 50 aa, MERS-CoV
PIIPGFGGDFNLTLLEPVSISTGSRSARSAIEDLLFDKVTIADPGYMQGY

SEQ ID NO: 7 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 587 aa, SARS-CoV-2
VASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 46 aa, SARS-CoV-2
PPIKDFGGFNFSQILPDPSKPSKRSFIEDLLFNKVTLADAGFIKQY

SEQ ID NO: 10 - protein, 50 aa, unknown
  Viral sequence, SARS-CoV, MERS-CoV, SARS-CoV-2 (Consensus Sequence)
  misc_feature (2)..(5): Xaa can be any naturally occurring amino acid
  misc_feature (9)..(9): Xaa can be any naturally occurring amino acid
  misc_feature (12)..(16): Xaa can be any naturally occurring amino acid
  misc_feature (18)..(27): Xaa can be any naturally occurring amino acid
  misc_feature (30)..(30): Xaa can be any naturally occurring amino acid
  misc_feature (37)..(37): Xaa can be any naturally occurring amino acid
  misc_feature (41)..(41): Xaa can be any naturally occurring amino acid
  misc_feature (44)..(44): Xaa can be any naturally occurring amino acid
  misc_feature (46)..(49): Xaa can be any naturally occurring amino acid
PXXXXFGGXFNXXXXXPXXXXXXXXXXRSXIEDLLFXKVTXADXGXXXXY

SEQ ID NO: 11 - protein, 8 aa, Homo sapiens
GYTFTSYA

SEQ ID NO: 12 - protein, 8 aa, Homo sapiens
INAGNGNT

SEQ ID NO: 13 - protein, 16 aa, Homo sapiens
ARDRHMVVPAAVFDNY

SEQ ID NO: 14 - protein, 25 aa, Homo sapiens [sequence omitted]

SEQ ID NO: 15 - protein, 17 aa, Homo sapiens
MHWVRQAPGQRLEWMGW

SEQ ID NO: 16 - protein, 38 aa, Homo sapiens
KYSQKFQGRVTITRDTSASTAYMELSSLRSEDTAVYYC

SEQ ID NO: 17 - protein, 11 aa, Homo sapiens
WGQGTLVTVSS

SEQ ID NO: 18 - protein, 122 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARDRHMVVPAAVFDYWGQGTLVTVSS

SEQ ID NO: 19 - protein, 6 aa, Homo sapiens
QSISSW

SEQ ID NO: 20 - protein, 3 aa, Homo sapiens
KAS

SEQ ID NO: 21 - protein, 5 aa, Homo sapiens
QQYGT

SEQ ID NO: 22 - protein, 26 aa, Homo sapiens
AIQLTQSPSTLSASVGDRVTITCRAS

SEQ ID NO: 23 - protein, 17 aa, Homo sapiens
LAWYQQKPGKAPKLLIY

SEQ ID NO: 24 - protein, 36 aa, Homo sapiens
SLESGVPSRFSGSGSGTEFTLTISSLQPDDFATYYC

SEQ ID NO: 25 - protein, 10 aa, Homo sapiens
FGQGTKWISN

SEQ ID NO: 26 - protein, 103 aa, Homo sapiens
AIQLTQSPSTLSASVGDRVTITCRASQSISSWLAWYQQKPGKAPKLLIYKASSLESGVPSRFSGSGSGTEFTLTISSLQPDDFATYYCQQYGTFGQGTKWISN

SEQ ID NO: 27 - protein, 10 aa, Homo sapiens
GGSISSSRHY

SEQ ID NO: 28 - protein, 7 aa, Homo sapiens
IDYSGGT

SEQ ID NO: 29 - protein, 16 aa, Homo sapiens
ARQVGHSGRGHNWFDP

SEQ ID NO: 30 - protein, 25 aa, Homo sapiens
LVQLQESGPRLVTPSETLSLTCTVS

SEQ ID NO: 31 - protein, 17 aa, Homo sapiens
WGWIRQPPGMGLEWIGS

SEQ ID NO: 32 - protein, 38 aa, Homo sapiens
YCNPSLKSRVTISEDTSKNQFSLKVNSVTAADTAVYYC

SEQ ID NO: 33 - protein, 11 aa, Homo sapiens
WGQGTLVTVSS

SEQ ID NO: 34 - protein, 124 aa, Homo sapiens
LVQLQESGPRLVTPSETLSLTCTVSGGSISSSRHYWGWIRQPPGMGLEWIGSIDYSGGTYCNPSLKSRVTISEDTSKNQFSLKVNSVTAADTAVYYCARQVGHSGRGHNWFDPWGQGTLVTVSS

SEQ ID NO: 35 - protein, 6 aa, Homo sapiens
QSIYNY

SEQ ID NO: 36 - protein, 3 aa, Homo sapiens
AAS

SEQ ID NO: 37 - protein, 9 aa, Homo sapiens
QQSYSSSVT

SEQ ID NO: 38 - protein, 26 aa, Homo sapiens [sequence omitted]

SEQ ID NO: 39 - protein, 17 aa, Homo sapiens
LNWYQQKPGKAPKFLIY

SEQ ID NO: 40 - protein, 36 aa, Homo sapiens
TLQTGVPSRFSGSGSGTDFTLTISSLQPEDFATYYC

SEQ ID NO: 41 - protein, 10 aa, Homo sapiens
FGQGTRLEIK

SEQ ID NO: 42 - protein, 107 aa, Homo sapiens
DIQMTQSPSSLSASVGDRVTITCRASQSIYNYLNWYQQKPGKAPKFLIYAASTLQTGVPSRFSGSGSGTDFTLTISSLQPEDFATYYCQQSYSSSVTFGQGTRLEIK

SEQ ID NO: 43 - protein, 8 aa, Homo sapiens
GFTFSSYA

SEQ ID NO: 44 - protein, 8 aa, Homo sapiens
ISGSGGST

SEQ ID NO: 45 - protein, 17 aa, Homo sapiens
AKAGNSKLRFFDWLLTM

SEQ ID NO: 46 - protein, 25 aa, Homo sapiens
QVQLVQSGGGLVQPGGSLRLSCAAS

SEQ ID NO: 47 - protein, 17 aa, Homo sapiens
MSWVRQTPGKGLEWVSA

SEQ ID NO: 48 - protein, 38 aa, Homo sapiens
YYADSVKGRFTISRDNSKNTLYLQMNSLRAADTAVYYC

SEQ ID NO: 49 - protein, 11 aa, Homo sapiens
WGQGTLVTVSS

SEQ ID NO: 50 - protein, 124 aa, Homo sapiens
QVQLVQSGGGLVQPGGSLRLSCAASGFTFSSYAMSWVRQTPGKGLEWVSAISGSGGSTYYADSVKGRFTISRDNSKNTLYLQMNSLRAADTAVYYCAKAGNSKLRFFDWLLTMWGQGTLVTVSS

SEQ ID NO: 51 - protein, 12 aa, Homo sapiens
QSVLYSSNNKNY

SEQ ID NO: 52 - protein, 3 aa, Homo sapiens
WAS

SEQ ID NO: 53 - protein, 9 aa, Homo sapiens
QQSYSSSVT

SEQ ID NO: 54 - protein, 26 aa, Homo sapiens
DIQLTQSPDSLAVSLGERATINCKSS

SEQ ID NO: 55 - protein, 17 aa, Homo sapiens
LAWYQQKPGQPPKLLIY

SEQ ID NO: 56 - protein, 36 aa, Homo sapiens
TRESGVPDRFSGSGSGTDFTLTISSLQAEDVAVYYC

SEQ ID NO: 57 - protein, 10 aa, Homo sapiens
FGPGTKVEIK

SEQ ID NO: 58 - protein, 127 aa, Homo sapiens
DIQLTQSPDSLAVSLGERATINCKSSDIQLTQSPDSLAVSLGERATINCKSSLAWYQQKPGQPPKLLIYWASTRESGVPDRFSGSGSGTDFTLTISSLQAEDVAVYYCQQYYSTPATFGPGTKVEIK

SEQ ID NO: 59 - protein, 8 aa, Homo sapiens
GFTFSSYD

SEQ ID NO: 60 - protein, 7 aa, Homo sapiens
IGTAGDT

SEQ ID NO: 61 - protein, 16 aa, Homo sapiens
GGPSVWLLLLLLRYGR

SEQ ID NO: 62 - protein, 24 aa, Homo sapiens
VQLVESGGGLVQPGGSLRLSCAAS

SEQ ID NO: 63 - protein, 17 aa, Homo sapiens
MHWVRQATGKGLEWVSA

SEQ ID NO: 64 - protein, 38 aa, Homo sapiens
YYPGSVKGRFTISRENAKNSLYLQMNSLRAGDTAVYYF

SEQ ID NO: 65 - protein, 11 aa, Homo sapiens
LGPRDHGHRLL

SEQ ID NO: 66 - protein, 111 aa, Homo sapiens
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYFGGPSVWLLLLLLRYGR

SEQ ID NO: 67 - protein, 9 aa, Homo sapiens
SSNIGAGYD

SEQ ID NO: 68 - protein, 3 aa, SARS
GNS

SEQ ID NO: 69 - protein, 11 aa, Homo sapiens
QSYDSSLSGSV

SEQ ID NO: 70 - protein, 25 aa, Homo sapiens
QSVLTQPPSVSGAPGQRVTISCTGS

SEQ ID NO: 71 - protein, 17 aa, Homo sapiens
VHWYQQLPGTAPKLLIY

SEQ ID NO: 72 - protein, 36 aa, Homo sapiens
NRPSGVPDRFSGSKSGTSASLAITGLQAEDEADYYC

SEQ ID NO: 73 - protein, 10 aa, Homo sapiens
FGGGTKLTVL

SEQ ID NO: 74 - protein, 111 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGGGTKLTVL

SEQ ID NO: 75 - skipped

SEQ ID NO: 76 - protein, 21 aa, SARS-CoV
IPFAMQMAYRFNGIGVTQNVL

SEQ ID NO: 77 - protein, 21 aa, MERS-CoV
IPFAQSIFYRLNGVGITQQVL

SEQ ID NO: 78 - protein, 21 aa, SARS-CoV-2
IPFAMQMAYRFNGIGVTQNVL

SEQ ID NO: 79 - protein, 21 aa, Unknown
  Viral sequence, SARS-CoV, MERS-CoV, SARS-CoV-2 (Consensus Sequence)
  misc_feature (5)..(8): Xaa can be any naturally occurring amino acid
  misc_feature (11)..(11): Xaa can be any naturally occurring amino acid
  misc_feature (16)..(16): Xaa can be any naturally occurring amino acid
  misc_feature (19)..(19): Xaa can be any naturally occurring amino acid
IPFAXXXXYRXNGIGXTQXVL

SEQ ID NO: 80 - protein, 18 aa, SARS-CoV
VSMAKTSVDCNMYICGDS

SEQ ID NO: 81 - protein, 18 aa, MERS-CoV
TTIQKVTVDCKQYVCNGF

SEQ ID NO: 82 - protein, 18 aa, SARS-CoV-2
VSMTKTSVDCTMYICGDS

SEQ ID NO: 83 - protein, 18 aa, Unknown
  Viral sequence, SARS-CoV, MERS-CoV, SARS-CoV-2 (Consensus Sequence)
  misc_feature (1)..(4): Xaa can be any naturally occurring amino acid
  misc_feature (11)..(12): Xaa can be any naturally occurring amino acid
  misc_feature (14)..(14): Xaa can be any naturally occurring amino acid
  misc_feature (16)..(18): Xaa can be any naturally occurring amino acid
XXXXKTSVDCXXYXCXXX

SEQ ID NO: 84 - protein, 44 aa, SARS-CoV
TIAIPTNFSISITTEVMPVSMAKTSVDCNMYICGDSTECANLLL

SEQ ID NO: 85 - protein, 44 aa, SARS-CoV-2
SIAIPTNFTISVTTEILPVSMTKTSVDCTMYICGDSTECSNLLL

SEQ ID NO: 86 - protein, 44 aa, MERS-CoV
KLSIPTNFSFGVTQEYIQTTIQKVTVDCKQYVCNGFQKCEQLLR

SEQ ID NO: 87 - protein, 36 aa, SARS-CoV
GAGAALQIPFAMQMAYRFNGIGVTQNVLYENQKQIA

SEQ ID NO: 88 - protein, 36 aa, SARS-CoV-2
GAGAALQIPFAMQMAYRFNGIGVTQNVLYENQKLIA

SEQ ID NO: 89 - protein, 36 aa, MERS-CoV
GLSSFAAIPFAQSIFYRLNGVGITQQVLSENQKLIA